SEQUENCE LISTING (4 sequences)

SEQ ID NO: 1 - DNA, 9009 bp, Homo sapiens
cagtgggtaagttccttaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggttcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccattggtcttaattctgaccaataaaaaaataagtcaggaggatgcaattgttgaaagctttgaaataaaataacatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgcaccccttcactatgactacacagaactctcctgatagtaaagggggctggaggcaaggataagttatagagcagttggaggaagcatccaaagactgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacataca

SEQ ID NO: 2 - protein, 2332 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 2114 aa, Sus scrofa
AIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAQNSRPPSASQKQFQTITSPEDDVELDPQSGERTQALEELSVPSGDGSMLLGQNPAPHGSSSSDLQEARNEADDYLPGARERNTAPSAAARLRPELHHSAERVLTPEPEKELKKLDSKMSSSSDLLKTSPTIPSDTLSAETERTHSLGPPHPQVNFRSQLGAIVLGKNSSHFIGAGVPLGSTEEDHESSLGENVSPVESDGIFEKERAHGPASLTKDDVLFKVNISLVKTNKARVYLKTNRKIHIDDAALLTENRASATFMDKNTTASGLNHVSNWIKGPLGKNPLSSERGPSPELLTSSGSGKSVKGQSSGQGRIRVAVEEEELSKGKEMMLPNSELTFLTNSADVQGNDTHSQGKKSREEMERREKLVQEKVDLPQVYTATGTKNFLRNIFHQSTEPSVEGFDGGSHAPVPQDSRSLNDSAERAETHIAHFSAIREEAPLEAPGNRTGPGPRSAVPRRVKQSLKQIRLPLEEIKPERGVVLNATSTRWSESSPILQGAKRNNLSLPFLTLEMAGGQGKISALGKSAAGPLASGKLEKAVLSSAGLSEASGKAEFLPKVRVHREDLLPQKTSNVSCAHGDLGQEIFLQKTRGPVNLNKVNRPGRTPSKLLGPPMPKEWESLEKSPKSTALRTKDIISLPLDRHESNHSIAAKNEGQAETQREAAWTKQGGPGRLCAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 4 - DNA, 6539 bp, Sus scrofa
ctaaaatcctttgtgaaaaatttggggcttttcacgaaatcagaaaaagtttaccttttctcccgggagtcaaaaatagcttagagaagaattcatctttcatttctccagctggacatttttgatcagtaagagtcatgcagctagagctctccacctgtgtctttctgtgtctcttgccactcggctttagtgccatcaggagatactacctgggcgcagtggaactgtcctgggactaccggcaaagtgaactcctccgtgagctgcacgtggacaccagatttcctgctacagcgccaggagctcttccgttgggcccgtcagtcctgtacaaaaagactgtgttcgtagagttcacggatcaacttttcagcgttgccaggcccaggccaccatggatgggtctgctgggtcctaccatccaggctgaggtttacgacacggtggtcgttaccctgaagaacatggcttctcatcccgttagtcttcacgctgtcggcgtctccttctggaaatcttccgaaggcgctgaatatgaggatcacaccagccaaagggagaaggaagacgataaagtccttcccggtaaaagccaaacctacgtctggcaggtcctgaaagaaaatggtccaacagcctctgacccaccatgtctcacctactcatacctgtctcacgtggacctggtgaaagacctgaattcgggcctcattggagccctgctggtttgtagagaagggagtctgaccagagaaaggacccagaacctgcacgaatttgtactactttttgctgtctttgatgaagggaaaagttggcactcagcaagaaatgactcctggacacgggccatggatcccgcacctgccagggcccagcctgcaatgcacacagtcaatggctatgtcaacaggtctctgccaggtctgatcggatgtcataagaaatcagtctactggcacgtgattggaatgggcaccagcccggaagtgcactccatttttcttgaaggccacacgtttctcgtgaggcaccatcgccaggcttccttggagatctcgccactaactttcctcactgctcagacattcctgatggaccttggccagttcctactgttttgtcatatctcttcccaccaccatggtggcatggaggctcacgtcagagtagaaagctgcgccgaggagccccagctgcggaggaaagctgatgaagaggaagattatgatgacaatttgtacgactcggacatggacgtggtccggctcgatggtgacgacgtgtctccctttatccaaatccgctcggttgccaagaagcatcccaaaacctgggtgcactacatctctgcagaggaggaggactgggactacgcccccgcggtccccagccccagtgacagaagttataaaagtctctacttgaacagtggtcctcagcgaattggtaggaaatacaaaaaagctcgattcgtcgcttacacggatgtaacatttaagactcgtaaagctattccgtatgaatcaggaatcctgggacctttactttatggagaagttggagacacacttttgattatatttaagaataaagcgagccgaccatataacatctaccctcatggaatcactgatgtcagcgctttgcacccagggagacttctaaaaggttggaaacatttgaaagacatgccaattctgccaggagagactttcaagtataaatggacagtgactgtggaagatgggccaaccaagtccgatcctcggtgcctgacccgctactactcgagctccattaatctagagaaagatctggcttcgggactcattggccctctcctcatctgctacaaagaatctgtagaccaaagaggaaaccagatgatgtcagacaagagaaacgtcatcctgttttctgtattcgatgagaatcaaagctggtacctcgcagagaatattcagcgcttcctccccaatccggatggattacagccccaggatccagagttccaagcttctaacatcatgcacagcatcaatggctatgtttttgatagcttgcagctgtcggtttgtttgcacgaggtggcatactggtacattctaagtgttggagcacagacggacttcctctccgtcttcttctctggctacaccttcaaacacaaaatggtctatgaagacacactcaccctgttccccttctcaggagaaacggtcttcatgtcaatggaaaacccaggtctctgggtcctagggtgccacaactcagacttgcggaacagagggatgacagccttactgaaggtgtatagttgtgacagggacattggtgattattatgacaacacttatgaagatattccaggcttcttgctgagtggaaagaatgtcattgaacccagaagctttgcccagaattcaagaccccctagtgcgagccaaaagcaattccaaaccatcacaagtccagaagatgacgtggagcttgacccgcagtctggagagagaacccaagcactggaagaactaagtgtcccctctggtgatgggtcgatgctcttgggacagaatcctgctccacatggctcatcctcatctgatcttcaagaagccaggaatgaggctgatgattatttacctggagcaagagaaagaaacacggccccatccgcagcggcacgtctcagaccagagctgcatcacagtgccgaaagagtacttactcctgagccagagaaagagttgaagaaacttgattcaaaaatgtctagttcatcagaccttctaaagacttcgccaacaattccatcagacacgttgtcagcggagactgaaaggacacattccttaggccccccacacccgcaggttaatttcaggagtcaattaggtgccattgtacttggcaaaaattcatctcactttattggggctggtgtccctttgggctcgactgaggaggatcatgaaagctccctgggagaaaatgtatcaccagtggagagtgacgggatatttgaaaaggaaagagctcatggacctgcttcactgaccaaagacgatgttttatttaaagttaatatctctttggtaaagacaaacaaggcacgagtttacttaaaaactaatagaaagattcacattgatgacgcagctttattaactgagaatagggcatctgcaacgtttatggacaaaaatactacagcttcgggattaaatcatgtgtcaaattggataaaagggccccttggcaagaaccccctaagctcggagcgaggccccagtccagagcttctgacatcttcaggatcaggaaaatctgtgaaaggtcagagttctgggcaggggagaatacgggtggcagtggaagaggaagaactgagcaaaggcaaagagatgatgcttcccaacagcgagctcacctttctcactaactcggctgatgtccaaggaaacgatacacacagtcaaggaaaaaagtctcgggaagagatggaaaggagagaaaaattagtccaagaaaaagtcgacttgcctcaggtgtatacagcgactggaactaagaatttcctgagaaacatttttcaccaaagcactgagcccagtgtagaagggtttgatggggggtcacatgcgccggtgcctcaagacagcaggtcattaaatgattcggcagagagagcagagactcacatagcccatttctcagcaattagggaagaggcacccttggaagccccgggaaatcgaacaggtccaggtccgaggagtgcggttccccgccgcgttaagcagagcttgaaacagatcagactcccgctagaagaaataaagcctgaaaggggggtggttctgaatgccacctcaacccggtggtctgaaagcagtcctatcttacaaggagccaaaagaaataacctttctttacctttcctgaccttggaaatggccggaggtcaaggaaagatcagcgccctggggaaaagtgccgcaggcccgctggcgtccgggaagctggagaaggctgttctctcttcagcaggcttgtctgaagcatctggcaaagctgagtttcttcctaaagttcgagttcatcgggaagacctgttgcctcaaaaaaccagcaatgtttcttgcgcacacggggatctcggccaggagatcttcctgcagaaaacacggggacctgttaacctgaacaaagtaaatagacctggaaggactccctccaagcttctgggtcccccgatgcccaaagagtgggaatccctagagaagtcaccaaaaagcacagctctcaggacgaaagacatcatcagtttacccctggaccgtcacgaaagcaatcattcaatagcagcaaaaaatgaaggacaagccgagacccaaagagaagccgcctggacgaagcagggagggcctggaaggctgtgcgctccaaagcctccggtcctgcgacggcatcagagggacataagccttcctacttttcagccggaggaagacaaaatggactatgatgatatcttctcaactgaaacgaagggagaagattttgacatttacggtgaggatgaaaatcaggaccctcgcagctttcagaagagaacccgacactatttcattgctgcggtggagcagctctgggattacgggatgagcgaatccccccgggcgctaagaaacagggctcagaacggagaggtgcctcggttcaagaaggtggtcttccgggaatttgctgacggctccttcacgcagccgtcgtaccgcggggaactcaacaaacacttggggctcttgggaccctacatcagagcggaagttgaagacaacatcatggtaactttcaaaaaccaggcgtctcgtccctattccttctactcgagccttatttcttatccggatgatcaggagcaaggggcagaacctcgacacaacttcgtccagccaaatgaaaccagaacttacttttggaaagtgcagcatcacatggcacccacagaagacgagtttgactgcaaagcctgggcctacttttctgatgttgacctggaaaaagatgtgcactcaggcttgatcggcccccttctgatctgccgcgccaacaccctgaacgctgctcacggtagacaagtgaccgtgcaagaatttgctctgtttttcactatttttgatgagacaaagagctggtacttcactgaaaatgtggaaaggaactgccgggccccctgccacctgcagatggaggaccccactctgaaagaaaactatcgcttccatgcaatcaatggctatgtgatggatacactccctggcttagtaatggctcagaatcaaaggatccgatggtatctgctcagcatgggcagcaatgaaaatatccattcgattcattttagcggacacgtgttcagtgtacggaaaaaggaggagtataaaatggccgtgtacaatctctatccgggtgtctttgagacagtggaaatgctaccgtccaaagttggaatttggcgaatagaatgcctgattggcgagcacctgcaagctgggatgagcacgactttcctggtgtacagcaaggagtgtcaggctccactgggaatggcttctggacgcattagagattttcagatcacagcttcaggacagtatggacagtgggccccaaagctggccagacttcattattccggatcaatcaatgcctggagcaccaaggatccccactcctggatcaaggtggatctgttggcaccaatgatcattcacggcatcatgacccagggtgcccgtcagaagttttccagcctctacatctcccagtttatcatcatgtacagtcttgacgggaggaactggcagagttaccgagggaattccacgggcaccttaatggtcttctttggcaatgtggacgcatctgggattaaacacaatatttttaaccctccgattgtggctcggtacatccgtttgcacccaacacattacagcatccgcagcactcttcgcatggagttgatgggctgtgatttaaacagttgcagcatgcccctgggaatgcagaataaagcgatatcagactcacagatcacggcctcctcccacctaagcaatatatttgccacctggtctccttcacaagcccgacttcacctccaggggcggacgaatgcctggcgaccccgggtgagcagcgcagaggagtggctgcaggtggacctgcagaagacggtgaaggtcacaggcatcaccacccagggcgtgaagtccctgctcagcagcatgtatgtgaaggagttcctcgtgtccagtagtcaggacggccgccgctggaccctgtttcttcaggacggccacacgaaggtttttcagggcaatcaggactcctccacccccgtggtgaacgctctggaccccccgctgttcacgcgctacctgaggatccaccccacgagctgggcgcagcacatcgccctgaggctcgaggttctaggatgtgaggcacaggatctctactga

(19) United States
(12) Patent Application Publication
(10) Pub. No.: US 2005/0123997 A1
(43) Pub. Date: Jun. 9, 2005

(54) Modified fVIII having reduced immunogenicity through mutagenesis of A2 and C2 epitopes

(76) Inventor: John S. Lollar, Decatur, GA (US)

Correspondence Address: Greenlee, Winner and Sullivan, P.C., Suite 201, 5370 Manhattan Circle, Boulder, CO, 80303, US

(21) Appl. No.: 10/973,941

(22) Filed: Oct. 25, 2004

Related U.S. Application Data
(60) Provisional application No. 60/516,647, filed on Oct. 30, 2003.

(51) Int. Cl. G01N 33/53; C07H 21/04; C12P 21/04; C07K 14/755

(52) U.S. Cl. 435/7.1; 435/69.6; 435/320.1; 435/325; 530/383; 536/23.5

(57) ABSTRACT

Specific amino acid loci of human fVIII interact with inhibitory antibodies of hemophilia patients after being treated with fVIII. Modified fVIII is disclosed in which the amino acid sequence is changed by multiple substitutions in human fVIII A2 and C2 domains. The modified fVIII is useful for hemophiliacs, either to avoid or prevent the action of inhibitory antibodies.

### CROSS-REFERENCE TO RELATED APPLICATIONS

This application claims the benefit of U.S. Provisional Application No. 60/516,647, filed Oct. 30, 2003, which is incorporated herein to the extent that there is no inconsistency with the present disclosure.

### STATEMENT REGARDING FEDERALLY SPONSORED RESEARCH OR DEVELOPMENT

Not Applicable

### BACKGROUND OF THE INVENTION

This invention relates generally to a modified mammalian factor VIII (“fVIII” herein) having amino acid substitutions which reduce its immunogenicity and/or antigenicity as compared to the proteins from which they were derived or other fVIII preparations such as human fVIII.

Blood clotting begins when platelets adhere to the cut wall of an injured blood vessel at a lesion site. Subsequently, in a cascade of enzymatically regulated reactions, soluble fibrinogen molecules are converted by the enzyme thrombin to insoluble strands of fibrin that hold the platelets together in a thrombus. At each step in the cascade, a protein precursor is converted by a protease that cleaves the next protein precursor in the series. Co-factors are required at most of the steps.

FVIII circulates as an inactive precursor in blood, bound tightly and non-covalently to von Willebrand factor. FVIII is proteolytically activated by thrombin and by factor Xa. Activation dissociates fVIII from von Willebrand factor and activates its procoagulant function in the cascade. In its active form, the protein fVIIIa is a cofactor that increases the catalytic efficiency of factor IXa toward factor X activation by several orders of magnitude.

People with deficiencies in fVIII (hemophilia A) or antibodies against fVIII who are not treated with fVIII suffer uncontrolled internal bleeding that may cause a range of serious symptoms, from inflammatory reactions in joints to early death. Severe hemophiliacs, who number about 10,000 in the United States, can be treated with infusion of human fVIII, which will restore the blood's normal clotting ability if administered with sufficient frequency and concentration. The functional definition of fVIII is that substance present in normal blood plasma that corrects the clotting defect in plasma derived from individuals with hemophilia A. Human fVIII has been characterized as a 2332 amino acid polypeptide, the amino acid sequence of which is given in SEQ ID NO: 2, having structural domains labeled as A1-A2-B-A3-C1-C2 (Vehar, G. A. et al., 1984, Nature, 312:337-342).

Although SEQ ID NO: 2 is the amino acid sequence of a human fVIII deduced from DNA sequencing from a human source, the term “human fVIII” is used herein to include a variety of sequence variations. For example, allelic variations in the amino-acid sequences of naturally-occurring proteins are well known and may be characterizing features of different populations or ethnic groups. Single nucleotide polymorphisms (SNP's) among individuals are well-recognized phenomena, some of which can result in coding changes and resulting variations in amino-acid sequence. In addition, it is known that many parts of the canonical fVIII sequence (SEQ ID NO: 2) can be deliberately changed by amino acid substitutions, by deleting segments of the canonical sequence, or by insertion into the canonical sequence. All or part of the B domain, from amino acids 741 to 1648, can be deleted without apparent loss of normal function. (Toole et al. (1986) Proc. Natl. Acad. Sci. USA 83(16):5939-5942; Eaton et al. (1986) Biochemistry 25(26):8343-8347; Langer et al. (1988) Behring Inst. Mitt. 82:16-25; Meulien et al. (1988) Protein Eng. 2(4):301-6; and U.S. Pat. No. 4,868,112, all of which are incorporated herein by reference.) Specific amino-acid substitutions can be made at many sites without significant loss of procoagulant activity. (U.S. Pat. Nos. 5,744,446; 5,859,204; 6,060,447; 6,180,371; 6,228,620; and 6,376,463.) Furthermore, comparisons between the amino acid sequences of human and animal fVIII's demonstrate a high degree of conservation of sequence in domains other than the B domain. (Diamond et al. (1992) Hum Mutat 1(3):248-57; Elder et al. (1993) Genomics 16(2):374-9; Healy et al. (1996) Blood 88(11):4209-14; Cameron et al. (1998) Thromb Haemost 79(2):317-22; Watzka et al. (2004) Thromb Haemost 91(1):38-42; and U.S. Pat. Nos. 5,364,771 and 5,859,204) Therefore, persons skilled in the art can reasonably predict where amino-acid substitutions are most likely to be tolerated in terms of coagulant activity, where substitutions of like-for-like amino acids can be made while retaining coagulant activity and where substitutions may result in loss of coagulant activity.

The term “human fVIII” as used herein, includes all such variations from the canonical sequence of SEQ ID NO: 2 as may exist, be discovered or artificially induced, provided the essential functional attribute of procoagulant activity in a human Hemophillia A patient, sufficient for therapeutic efficacy, is maintained. Procoagulant activity is commonly expressed in units/ml of plasma, measured by either a coagulation assay or a chronogenic assay comparing a sample of a patient's plasma with an international reference standard. The activity of normal plasma is about 1 unit/ml. A “human fVIII” as defined herein will have procoagulant activity to provide at least 0.01 units/ml plasma 15 minutes after administration to a hemophiliac patient lacking inhibitor antibodies. Although the number of changes from the canonical sequence is likely to be small, human fVIII, as herein defined, can be distinguished from the other animal fVIII's by simple sequence identity comparison. Thus, a given sequence of human fVIII will have more amino acids identical to canonical SEQ ID NO: 2 than to any known animal sequence, after taking into account corresponding deletions, e.g. in the B domain.

The development of antibodies (“inhibitors” or “inhibitory antibodies”) that inhibit the activity of fVIII is a serious complication in the management of patients with hemophilia. Inhibitory antibodies (inhibitors) to fVIII either develop as alloantibodies in hemophilia A patients following fVIII infusions or as autoantibodies in nonhemophiliacs (Hoyer, L. W. and D. Scandella, 1994, Semin. Hematol. 31:1-5). Antibodies develop in approximately 20% of patients with hemophilia A in response to therapeutic infusions of fVIII. In previously untreated patients with hemophilia A who develop inhibitors, the inhibitors usually develop within one year of treatment. Additionally, autoantibodies that inactivate fVIII occasionally develop in individuals with previously normal fVIII levels. Antibodies to epitopes in the A2, ap-A3, and C2 domains within the A1-A2-B-ap-A3-C1-C2 fVIII molecule are responsible for all anticoagulant activity in most inhibitor plasmas (Prescott, R. et al., 1997, Blood 89:3663-3671; Barrow, R. T. et al., 2000, Blood 95:557-561).

Anti-A2 inhibitors inhibit the function of activated fVIII within the intrinsic pathway factor X activation complex, apparently by blocking the binding of factor X to the complex (Lollar, P. et al., 1994, J. Clin. Invest. 93:2497-2504). The A2 epitope has been localized to a single, continuous sequence bounded by residues R484-I508 (Healey, J. F. et al., 1995, J. Biol. Chem. 270:14505-14509).

The 18-kDa C2 domain, defined as residues Ser2173-Tyr2332 in single chain human fVIII, contains a phospholipid membrane-binding site that is necessary for the normal procoagulant function of fVIII. Human C2-specific anti-fVIII antibodies inhibit this interaction (Arai, M. et al., 1989, J. Clin. Invest. 83:1978-1984). Consistent with this, binding to phospholipids protects fVIII from inactivation by fVIII inhibitors (Arai et al., supra; Barrowcliffe, T. W. et al., 1983, J. Lab. Clin. Med. 101:34-43). The C2 domain also contains part of the von Willebrand factor (vWf) binding site (Saenko, E. L. et al., 1994, J. Biol. Chem. 269:11601-11605; Saenko, E. L. and Scandella, D., 1997, J. Biol. Chem. 272:18007-18014). Some inhibitors may act by interfering with this interaction (Shima, M. et al., 1995, Br. J. Haematol. 91:714-721; Saenko, E. L. et al., 1996 J. Biol. Chem. 271:27424-27431; Gilles, J. G. et al., 1999, Thromb. Haemost. 82:40-45).

Patients who develop antibodies to human fVIII can be managed by increasing the dose of fVIII provided the inhibitor titer is low enough. However, often the inhibitor titer is so high that it cannot be overwhelmed by increased amounts of fVIII. An alternative strategy is to bypass the need for fVIII during normal hemostasis using factor IX complex preparations (for example, KONYNE®, Proplex®) or using recombinant human fVIIIa. Additionally, since porcine fVIII usually has substantially less reactivity with inhibitors than human fVIII, a partially purified porcine fVIII preparation (HYATE:C®) is used. Many patients who have developed inhibitory antibodies to human fVIII have been successfully treated with porcine fVIII and have tolerated such treatment for long periods of time. However, administration of porcine fVIII is not a complete solution because inhibitors occasionally develop to porcine fVIII after one or more infusions.

Hybrid fVIII molecules which substitute regions of human fVIII with the corresponding regions from animals are well known in the art. U.S. Pat. No. 5,888,974 (Lollar et al.) discloses hybrid procoagulant fVIII produced by the isolation and recombination of human with non-human fVIII subunits or domains. Similarly, U.S. Pat. Nos. 5,663,060 and 5,583,209 describe hybrid fVIII comprising combinations of non-human and human heavy chain and light chain subunits. U.S. Pat. No. 5,364,771 describes purified hybrid fVIII comprised of human and porcine combinations of the heavy and light subunits, including a human fVIII with a porcine A2 domain substituted for the human A2 domain.

Several preparations of human plasma-derived fVIII of varying degrees of purity are available commercially for the treatment of hemophilia A. These include a partially-purified fVIII derived from the pooled blood of many donors that is heat- and detergent-treated to inactivate viruses but contain a significant level of antigenic proteins; and a monoclonal antibody-purified fVIII that has lower levels of antigenic impurities and viral contamination. Another alternative product is recombinant human fVIII (currently sold under the trade name Refacto®) which would be free of viral contaminants. Unfortunately, human fVIII is unstable at physiologic concentrations and pH, is present in blood at an extremely low concentration (0.2 μg/ml plasma), and has low specific clotting activity. A significant proportion of patients receiving recombinant human fVIII develop inhibitory antibodies to that product.

Hemophiliacs require daily replacement of fVIII to prevent bleeding and the resulting deforming hemophilic arthropathy. However, supplies have been inadequate and problems in therapeutic use occur due to difficulty in isolation and purification, immunoreactivity, and the necessity of removing the AIDS and hepatitis infectivity risk. The use of recombinant human fVIII, partially-purified porcine fVIII, or human-animal hybrid fVIII will not resolve all the problems.

The problems associated with the commonly used, commercially available, plasma-derived fVIII have stimulated significant interest in the development of a better fVIII product. There is a need for fVIII product having greater specific activity so that more units of clotting activity can be delivered per milligram of total protein; a fVIII molecule that is stable at a selected pH and physiologic concentration; a fVIII molecule that is less apt to cause production of inhibitory antibodies; and a fVIII molecule that evades immune detection in patients who have already acquired antibodies to human fVIII.

Reduction of antigenicity (inhibition by reaction with antibodies) is described in U.S. Pat. Nos. 6,180,371 and 5,744,446 which describe modified fVIII having amino acid substitutions in the A2 domain. U.S. Pat. Nos. 6,376,463 and 5,859,204 disclose site specific replacement of amino acids in the 484-509 region in the A2 domain of human fVIII. These patents do not disclose or suggest specific amino acid substitutions in the C2 domain which reduce antigenicity or immunogenicity as compared to wild-type fVIII or the corresponding recombinant fVIII. These patents also do not disclose the specific triple mutant in the A2 domain described in the present invention.

U.S. Pat. No. 6,770,744 discloses modified fVIII with site specific amino acid substitutions in the C2 domain at positions 2199, 2200, 2223, 2227, 2251, 2252 relative to human fVIII. The substitutions are disclosed to reduce antigenicity with respect to certain antibodies directed to the C2 domain. The U.S. Pat. No. 6,770,744 patent discloses a quadruple mutant having amino acid substitutions at each of positions 2199, 2200, 2223 and 2227, but does not disclose the specific quadruple mutant described in the present invention.

Pratt et al. (1999, Nature 402:439-442) have reported an X-ray crystallography structure of the C2 domain of human fVIII at 1.5A resolution. Pratt et al. reported that the structure partly explains why mutations in the C2 region of fVIII lead to bleeding disorders. In fact, 21 residues in the C2 region were reported to be sites of deleterious point mutations in patients with hemophilia A. Shima et al. report C2 directed inhibitory antibodies that interfere with fVIII with respect to phospholipid and Von Willebrand factor binding. Thus, it is taught by Pratt et al. that C2 inhibitors, i.e., those related to some bleeding disorders in individuals with hemophilia A, interfere with the binding of the C2 domain to phospholipid and Von Willebrand factor. This conclusion, combined with their determination that M2199, F2200, V2223, K2227, L2251 and L2252 appear at the protein-phospholipid interface, suggests that these amino acids are important for normal fVIII activity and mutation of these residues would lead to detrimentally altered phospholipid and/or Von Willebrand binding along with an associated increase in bleeding disorders. However, U.S. Pat. No. 6,770,744 discloses amino acid substitutions at amino acid positions M2199, F2200, V2223, K2227, L2251 and L2252 which result in reduced antigenicity while maintaining coagulant activity.

U.S. Pat. Nos. 6,180,371; 5,888,974; 5,859,204; 5,744,446; 5,663,060; 5,583,209; and 5,364,771 and U.S. patent application Ser. No. 10/131,510 (all of which are incorporated herein by reference) do not disclose the specific triple mutant in the A2 domain described in the present invention. Nor do they disclose amino acid substitutions in the C2 domain of fVIII which reduce the immunogenicity as compared to wild-type fVIII or the corresponding recombinant fVIII. U.S. Pat. No. 6,770,744 (also incorporated herein by reference) discloses other amino acid substitutions in the C2 domain, but does not disclose specific amino acid substitutions in the A2 domain for reducing immunogenicity, nor the specific quadruple mutant in the C2 domain described in the present invention. None of the above patents or applications discloses a mutant combining site specific amino acid substitutions in both the A2 and C2 domain.

It is not clear from previous studies which amino acid residues and corresponding substitutions would lead to improved fVIII molecules. As described further below, one modified fVIII that contains the R484A/R489A mutation has no significant difference in immunogenicity compared to normal human fVIII, while a different fVIII that contains the R484A/R489A/P492A mutant has significantly lower immunogenicity than normal human fVIII. Similarly, three specific fVIII mutants described below, designated A2C2epi1, A2C2epi2 and A2C2epi3, have the R484A/R489A/P492A mutant in the A2 domain combined with additional multiple amino acid substitutions in C2 domain. The amino acid substitutions in the C2 domain differ between A2C2epi1, A2C2epi2, and A2C2epi3 by no more than two amino acids. Despite the fact that all three of these fVIII mutants contain the R484A/R489A/P492A mutant, which has lower immunogenicity than normal human fVIII, one fVIII mutant, A2C2epi2, does not have reduced immunogenicity compared to normal human fVIII. The other two mutants have lower immunogenicity than normal human fVIII, but only the A2C2epi3 fVIII mutant has significantly lower immunogenicity than the fVIII mutant having only the R484A/R489A/P492A mutant.

It is an object of the present invention to provide a fVIII that corrects hemophilia in a patient deficient in fVIII and can be administered routinely with a reduced risk of developing inhibitory antibodies to fVIII compared to currently available products. It is a further object of the present invention to provide methods for treatment of hemophiliacs. It is still another object of the present invention to provide a fVIII that is stable at a selected pH and physiologic concentration. It is yet another object of the present invention to provide a fVIII that has greater or more prolonged specific coagulant activity than human fVIII.

### SUMMARY OF THE INVENTION

The present invention generally relates to compositions comprising recombinant human fVIII. The compositions of the invention comprise isolated, purified recombinant human fVIII molecules with coagulant activity wherein the recombinant fVIII has amino acid substitutions in the A2 and C2 domains which reduce immunogenicity and optionally antigenicity as compared to the proteins from which they were derived or other fVIII preparations. DNA sequences encoding the novel compositions of the invention as well as methods of producing the novel compositions comprising fVIII are also provided. Methods of treating patients in need of treatment with fVIII are also within the scope of this invention.

A first embodiment of the invention provides a modified fVIII comprising the C2 domain of human fVIII with a quadruple amino acid substitution in the C2 domain. The amino acid substitutions in the C2 domain of the modified recombinant fVIII reduce its capacity to elicit inhibitory antibodies as compared to the proteins from which they were derived or other available fVIII preparations. The novel composition of this embodiment is a modified fVIII molecule having amino acid substitutions in the C2 domain at each of positions 2199, 2200, 2251 and 2252.

A further embodiment is a modified fVIII having the quadruple mutant M2199L/F2200L/L2251V/L2252F (leucine is substituted for methionine 2199, leucine is substituted for phenylalanine 2200, valine is substituted for leucine 2251, and phenylalanine is substituted for leucine 2252). M2199L/F2200L/L2251V/L2252F is referenced to the human fVIII numbering system wherein amino acid number 1 is the amino terminal alanine of mature fVIII. Amino acids will be identified herein using the generally accepted single letter code.

In one embodiment, the fVIII having the quadruple mutant comprises the amino acid sequence set forth in SEQ ID NO: 2 from amino acids 2173-2332. In another embodiment, the fVIII having the quadruple mutant comprises the amino acid sequence set forth in SEQ ID NO: 2 from amino acids 1649-2332. In another embodiment, the fVIII having the quadruple mutant comprises the amino acid sequence set forth in SEQ ID NO: 2 from amino acids 1-740 and 1649-2332. In another embodiment, the fVIII having the quadruple mutant comprises the amino acid sequence set forth in SEQ ID NO: 2 from amino acids 1-745 and 1640-2332. In another embodiment, the fVIII having the quadruple mutant comprises the amino acid sequence set forth in SEQ ID NO: 2 from amino acids 1-2332. The modified fVIII has reduced immunogenicity and antigenicity to an inhibitory antibody as compared to unmodified fVIII and may lack part or the entire B domain. The modified fVIII comprises an A1/A2/A3-C1-C2 heterotrimer, an A1-A2/A3-C1-C2 heterodimer, or a single continuous polypeptide. This invention also provides pharmacological compositions comprising the above modified fVIII. This invention also provides DNA encoding the above modified fVIII and methods of making the modified fVIII by expressing said DNA. The corresponding nucleotide sequence encoding human fVIII is disclosed in SEQ ID NO: 1.

One embodiment of the present invention is a polypeptide comprising an amino acid sequence having the quadruple mutant M2199L/F2200L/L2251V/L2252F and having at least about 85% sequence homology, more usually at least about 95% sequence homology, with the C2 domain (amino acids 2173-2332) set forth in SEQ ID NO: 2.

U.S. Pat. No. 6,770,744 discloses substitutions at positions homologous to human fVIII including, but not limited to, M2199, F2200, V2223, K2227, L2251, and L2252. Of particular note, U.S. Pat. No. 6,770,744 discloses substituting isoleucine for M2199. However, a modified fVIII having the quadruple mutant M21991/F2200L/L2251V/L2252F, as disclosed in the U.S. Pat. No. 6,770,744 patent, did not have significantly lower immunogenicity compared to normal human fVIII. In contrast, a modified fVIII having the novel composition of this embodiment, the quadruple mutant M2199L/F2200L/L2251V/L2252F, which substitutes leucine for M2199 instead of isoleucine, does have significantly reduced immunogenicity compared to normal human fVIII and retains coagulant activity.

Another embodiment of this invention provides a modified fVIII comprising the A2 domain of human fVIII with a triple amino acid substitution in the A2. The amino acid substitutions in the A2 domain of the modified fVIII reduce its immunogenicity compared to the proteins from which they were derived or other available fVIII preparations. The novel composition of this embodiment is a modified fVIII having immunoreactivity reducing amino acid substituted in the A2 domain at each of positions 484, 489 and 492.

A further embodiment is a modified fVIII having the triple mutant R484A/R489A/P492A, where alanine is substituted for arginine 484, arginine 489, and proline 492. R484A/R489A/P492A is referenced to the human fVIII numbering system wherein amino acid number 1 is the amino terminal alanine of mature fVIII.

A further embodiment is a modified fVIII having the corresponding porcine amino acids substituted for arginine 484, arginine 489, and proline 492. The amino acid and cDNA sequences of porcine fVIII are set forth in SEQ ID NO: 3 and SEQ ID NO: 4, respectively. In SEQ ID NO: 4, the coding region begins at nucleotide position 195, the triplet GCC being the codon for amino acid number 1 (Ala) of the mature protein as given in SEQ ID NO: 3.

In one embodiment, the fVIII having the triple mutant comprises the amino acid sequence set forth in SEQ ID NO: 2 from amino acids 373-740. In another embodiment, the fVIII having the triple mutant comprises the amino acid sequence set forth in SEQ ID NO: 2 from amino acids 1-740. In another embodiment, the fVIII having the triple mutant comprises the amino acid sequence set forth in SEQ ID NO: 2 from amino acids 1-740 and 1649-2332. In another embodiment, the fVIII having the triple mutant comprises the amino acid sequence set forth in SEQ ID NO: 2 from amino acids 1-745 and 1640-2332. In another embodiment, the fVIII having the triple mutant comprises the amino acid sequence set forth in SEQ ID NO: 2 from amino acids 1-2332. The modified fVIII has reduced immunogenicity and antigenicity to an inhibitory antibody as compared to unmodified fVIII and may lack part or the entire B domain. The modified fVIII comprises an A1/A2/A3-C1-C2 heterotrimer, an A1-A2/A3-C1-C2 heterodimer, or a single continuous polypeptide. This invention also provides pharmacological compositions comprising the above modified fVIII. This invention also provides DNA encoding the above modified fVIII and methods of making the modified fVIII by expressing said DNA. The corresponding nucleotide sequence encoding human fVIII is disclosed in SEQ ID NO 1.

One embodiment of the present invention is a polypeptide comprising an amino acid sequence having the triple mutant R484A/R489A/P492A and having at least about 85% sequence homology, more usually at least about 95% sequence homology, with the A2 domain (amino acids 373-740) set forth in SEQ ID NO: 2.

Another embodiment of this invention provides a modified fVIII comprising the A2 and C2 domains of human fVIII with a triple amino acid substitution in the A2 domain combined with the previously described quadruple amino acid substitution in the C2 domain. The amino acid substitutions in both the A2 and C2 domains of the modified fVIII reduce immunogenicity of the modified fVIII when compared to the proteins from which they were derived or other available fVIII preparations having amino-acid substitutions in either the A2 domain or C2 domain. The novel composition of this embodiment is modified fVIII molecule having amino acid substitutions in the C2 domain at each of positions 2199, 2200, 2251 and 2252, and having amino acid substitutions in the A2 domain at each of positions 484, 489 and 492.

A further embodiment of the present invention is a modified human fVIII molecule having the mutation R484A/R489A/P492A/M2199L/F2200L/L2251V/L2252F, where alanine is substituted for arginine 484, arginine 489, and proline 492, leucine is substituted for methionine 2199, leucine is substituted for phenylalanine 2200, valine is substituted for leucine 2251, and phenylalanine is substituted for leucine 2252.

In one embodiment, the fVIII having the R484A/R489A/P492A/M2199L/F2200L/L2251V/L2252F mutation comprises the amino acid sequence set forth in SEQ ID NO: 2 from amino acids 373-740 and 2173-2332. In another embodiment, the fVIII having the mutation comprises the amino acid sequence set forth in SEQ ID NO: 2 from amino acids 1-740 and 1649-2332. In another embodiment, the fVIII having the mutant comprises the amino acid sequence set forth in SEQ ID NO: 2 from amino acids 1-745 and 1640-2332. In another embodiment, the fVIII having the mutation comprises the amino acid sequence set forth in SEQ ID NO: 2 from amino acids 1-2332. The modified fVIII has reduced immunogenicity and antigenicity to an inhibitory antibody as compared to unmodified fVIII and may lack part or the entire B domain. The modified fVIII comprises an A1/A2/A3-C2 heterotrimer, an A1-A2/A3-C1-C2 heterodimer, or a single continuous polypeptide. This invention also provides pharmacological compositions comprising the above modified fVIII. This invention also provides DNA encoding the above modified fVIII and methods of making the modified fVIII by expressing said DNA. The corresponding nucleotide sequence encoding human fVIII is disclosed in SEQ ID NO: 1.

One embodiment of the present invention is a polypeptide comprising an amino acid sequence having the mutant R484A/R489A/P492A/M2199L/F2200L/L2251V/L2252F and having at least about 85% sequence homology, more usually at least about 95% sequence homology, with the A2 domain (amino acids 373-740) and C2 domain (amino acids 2173-2332) set forth in SEQ ID NO: 2.

In one embodiment of the present invention, the modified fVIII comprises the A1, A2, A3, C1 and C2 domains of human fVIII. In another embodiment of the present invention, the modified fVIII comprises the C2 domain of porcine fVIII and the A2 domain of human fVIII having the above mentioned amino acid substitutions at arginine 484, arginine 489, and proline 492. In another embodiment, the modified fVIII comprises the A2 domain of porcine fVIII and the C2 domain of human fVIII having the previously described quadruple amino acid substitution in the C2 domain. The amino acid sequence for porcine fVIII is set forth in SEQ ID NO: 3.

Another embodiment of the invention provides DNA sequences comprising coding sequences for the modified fVIII of the invention. Yet another embodiment of the invention provides methods of producing the modified fVIII of the invention.

fVIII of the present invention may exist as a heterodimer or heterotrimer. As a result, the separate domains and subunits do not necessarily have to be expressed from the same DNA segment. For example, U.S. Pat. Nos. 6,060,447 and 6,228,620 disclose separately expressing a fVIII heavy chain (amino acids 1-740) and a fVIII light chain (amino acids 1649-2332). One embodiment of the present invention is a DNA complex comprising: (a) a first DNA segment encoding the A2 domain or A1 and A2 domains where alanine is substituted for arginine 484, arginine 489, and proline 492; and (b) a second DNA segment encoding the C2 domain, or A3, C1, and C2 domains where leucine is substituted for methionine 2199, leucine is substituted for phenylalanine 2200, valine is substituted for leucine 2251, and phenylalanine is substituted for leucine 2252.

The invention provides a method for reducing the immunogenicity of fVIII as well as a recombinant fVIII having reduced immunogenicity produced by the method. In particular, modified human fVIII, and methods of making such molecules, having immunoreactivity reducing amino acid substitutions in the A2 and C2 domain are described. “Immunoreactivity reducing” amino acids are defined herein as those amino acids which do not significantly contribute to an antigen-antibody interaction. Non-limiting examples of some amino acids known to be immunoreactivity-reducing include alanine, methionine, leucine, serine, and glycine. It will be understood that the reduction of immunoreactivity achievable by a given amino acid substitution will also depend on any effects the substitution may have on protein conformation, epitope accessibility and the like.

Also provided are pharmaceutical compositions and methods for treating patients having fVIII deficiency comprising administering modified recombinant fVIII.

### BRIEF DESCRIPTION OF THE DRAWINGS

FIG. 1 shows diagrams of various FVIII constructs and their structural domains. Human, porcine and murine forms of B domain deleted fVIII are designated HBD, PBD, and MBD, respectively. Bold type in the amino acid alignments corresponds to amino acid mutagenesis or substitution within the 484-508 A2 epitope that differs from human fVIII. Included are amino acid sequences of the A2 epitope for human B domain deleted constructs having a double amino acid substitution or a triple amino acid substitution. Also shown is a human construct designated HP9 where the 484-508 A2 epitope is substituted with the corresponding porcine amino acid sequence. Also shown is a murine construct designated HM1 where the 484-508 A2 epitope is substituted with the corresponding human amino acid sequence.

FIG. 2 shows the antigenicity of the human R484-I508 A2 segments in human B domain deleted (HBD) immunized hemophilia A mice. Plasmas were obtained from hemophilia A mice immunized with HBD and were assayed for inhibitory anti-fVIII antibodies against HBD and HP9 by Bethesda assay (A) and anti-fVIII antibodies against MBD and HM1 by ELISA (B) as described in Example 1. Each data pair corresponds to individual mouse plasma from an HBD-immunized mouse.

FIG. 3 shows the correlation between the reduced antigenicity of HP9 and the increased antigenecity of HM1 in HBD-immunized hemophilia A mice. Ratios of assay titers for HBD to HP9 and for HM1 to MBD from the experiment shown in FIG. 2 were calculated and plotted. The regression line corresponds to a coefficient of correlation, r, of 0.61, which is significantly greater than zero (p=0.001, t test).

FIG. 4 shows the comparative immugenicity of HBD, R484A/R489A/P492A and R484A/R489A. Hemophilia A mice received intravenous injections of HBD, the triple mutant R484A/R489A/P492A or the double mutant R484A/R489A and then were tested for inhibitory anti-fVIII antibodies by Bethesda assay as described in Materials and Methods. The horizontal lines represent the sample means. The statistical parameters of the samples (mean±s.d.) were 670±500, 320±310, and 780±570 for the HBD, R484A/R489A/P492A, and R484A/R489A groups, respectively.

FIG. 5 shows in vivo clearance of R484A/R489A/P492A and HBD. Mice were injected with 100 U/kg R484A/R489A/P492A (open circles) or HBD (closed circles) by tail vein and assayed for fVIII activity by chromogenic assay described in Materials and Methods. A different group of three mice was used at each time point. Data represent the mean and standard deviation.

FIG. 6 shows candidate low immunogenicity fVIII C2 domain mutations. The amino acid substitutions at phospholipid binding loops 1 and 3 of the C2 epi1, C2 epi2 and C2 epi3 mutants are shown compared to the native human amino acids.

FIG. 7 shows the expression of fVIII constructs from BHK-derived cells. FVIII coagulant activity from triple-flask supernatants was assayed at the indicated times during the production runs. In this figure C2epi1, C2epi2 and C2epi3 refer to A2C2epi1, A2C2epi2 and A2C2epi3, respectively.

FIG. 8 shows the SDS-PAGE of fVIII constructs. Purified HBD, A2epi7, A2C2epi1, A2C2epi2 and A2C2epi3 (1.3 μg), with and without treatment thrombin (IIa), underwent 4-15% gradient SDS-PAGE, followed by Gel-Code Blue staining. SC, single chain; H, heavy chain (A1-A2); LC, light chain (ap-A3-C1-C2); LCIIa, thrombin-cleaved light chain (A3-C1-C2); *, low molecular weight contaminant. In this figure C2epi1, C2epi2 and C2epi3 refer to A2C2epi1, A2C2epi2 and A2C2epi3, respectively.

FIG. 9 shows the Bethesda assay of the comparative immunogenecity of HBD, A2epi7, A2C2epi1, A2C2epi2 and A2C2epi3 in hemophilia mice. Mice received intravenous injections of fVIII preparations and then were tested for inhibitory anti-fVIII antibodies by Bethesda assay as described in Example 2. The horizontal lines represent the sample means.

FIG. 10 shows the ELISA analysis of comparative immunogenicity of HBD, A2epi7, A2C2epi1, A2C2epi2 and A2C2epi3. Mice received intravenous injections of fVIII preparation and then were tested by Elisa as described in Example 2. The horizontal lines represent the sample means.

FIG. 11 shows the single human domain hybrid human/porcine fVIII constructs. The shaded areas designate domains having amino acid sequences corresponding to porcine fVIII. The white areas designate domains having amino acid sequences corresponding to human fVIII.

FIG. 12 shows the domain specific ELISA titers from mice immunized with HBD, A2epi7 or A2C2epi3. Plasmas from ten mice selected from each of the HBD and A2epi7 groups and all five ELISA positive mice in the A2C2epi3 group were subjected to ELISA on single human domain hybrid human/porcine fVIII coated plates as described in Example 2.

FIG. 13 shows the in vitro recovery of HBD, A2epi7, A2C2epi1, A2C2epi2 and A2C2epi3. Human hemophilia A plasma was reconstituted with purified HBD, A2epi7, A2C2epi1, A2C2epi2 and A2C2epi3 followed by assay of fVIII coagulant as described in Example 2. Data are expressed as means and sample standard deviations of expected recovery based on the nominal concentrations of purified proteins and result from at least eight samples done on separate days.

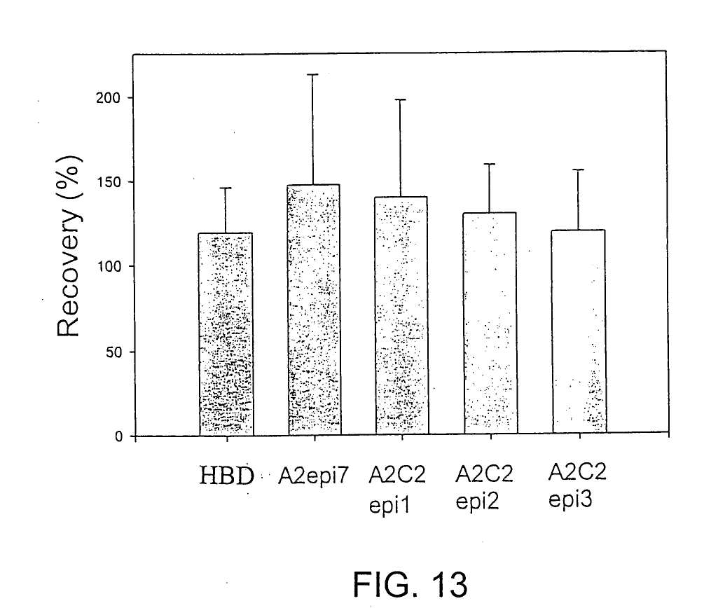

FIG. 14 shows the concentration dependence of apt-reagent on the clotting times of human hemophilia A plasma reconstituted with HBD and A2C2epi3. Human hemophilia A plasma was reconstituted with HBD or A2C2epi3 and clotting times were determined as in standard one-stage fVIII assays, except the concentration of a PTT-reagent was varied. The data represents means and ranges of two independent experiments in which duplicate samples were assayed.

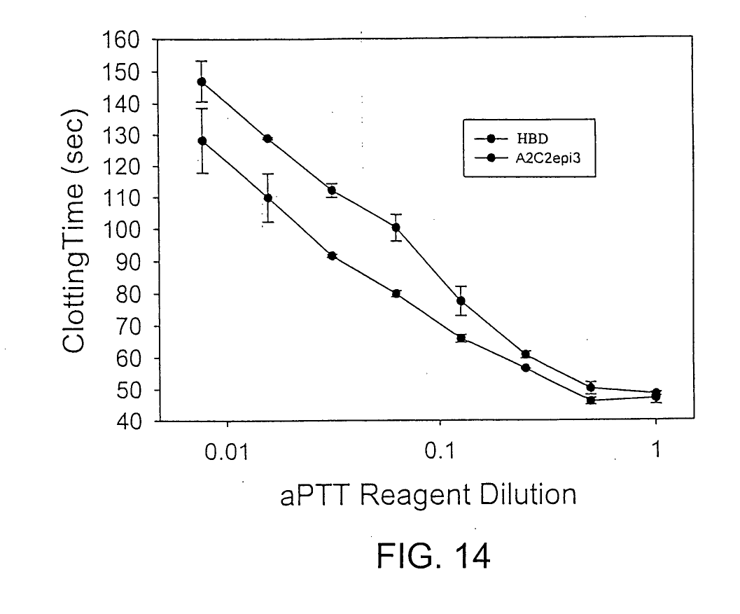

### DETAILED DESCRIPTION OF THE INVENTION

The present invention generally relates to compositions comprising recombinant human fVIII. The compositions of the present invention comprise isolated, purified recombinant human fVIII molecules with coagulant activity. It was previously discovered that mutations in the C2 domain of fVIII reduced the binding of inhibitory antibodies of the mutants as compared to the proteins from which they were derived and/or other fVIII preparations. Novel compositions of the invention comprise recombinant human fVIII with specific amino acid substitutions in the C2 domain which reduce immunogenicity or antigenicity as compared to the proteins from which they were derived or other available fVIII preparations. Furthermore, it has been previously discovered that mutations in the A2 domain of fVIII reduce the binding of inhibitory antibodies of the mutants as compared to the proteins from which they were derived and/or other fVIII preparations. Novel compositions of the invention comprise recombinant factor human VIII with specific amino acid substitutions in the A2 domain which reduce immunogenicity or antigenicity as compared to the proteins from which they were derived or other available fVIII preparations. Novel compositions of the invention also comprise recombinant human fVIII with specific amino acid substitutions in both the A2 and C2 domains which reduce immunogenecity or antigenicity as compared to the proteins from which they were derived or other available fVIII preparations.

Related embodiments of the invention provide for methods of treating patients in need of fVIII treatment, methods of producing the novel recombinant fVIII compositions of the invention, DNA sequences comprising coding sequences of the novel recombinant fVIII proteins, and pharmaceutical compositions comprising the novel fVIII proteins.

The present invention further provides active recombinant hybrid fVIII molecules or fragments thereof, the nucleic acid sequences encoding these hybrids, methods of preparing and isolating them, and methods for characterizing them. These hybrids comprise human/animal, animal/animal, or other such hybrid fVIII molecules, and further comprise at least one specific amino acid sequence in the A2 and/or C2 domain having one or more unique amino acids of the fVIII of one species substituted for the corresponding amino acid sequence (or amino acid) of the fVIII of the other species; or comprises at least one sequence in the A2 and/or C2 domain including one or more amino acids having no known sequence identity to fVIII substituted for specific amino acid sequence in human, animal, or hybrid fVIII. The resulting recombinant hybrid fVIII has reduced or no immunoreactivity to fVIII inhibitory antibodies, compared to human or porcine fVIII.

Unless otherwise specified or indicated, as used herein, “fVIII” denotes any functional fVIII protein molecule from human, any animal, any hybrid fVIII or any modified fVIII. “Hybrid fVIII” or “modified fVIII” denotes any functional fVIII protein, molecule or fragment thereof comprising fVIII amino acid sequences from one species substituted with one or more amino acids from another species, or with one or more amino acids having no known sequence identity with human or animal fVIII. Such hybrid and modified combinations include a fVIII amino acid sequence of human origin substituted with an amino acid sequence from an animal fVIII or an amino acid sequence having no known sequence identity to human or animal fVIII. Such combinations also include a fVIII amino sequence derived from more than two species, such as human/pig/mouse, or from one or more species in which an amino acid sequence having no known sequence identity to fVIII is substituted. Unless otherwise indicated, “hybrid fVIII” and “modified fVIII” include fragments of the fVIII, which can be used as probes for research purposes or as diagnostic reagents.

A “fusion protein” or “fusion fVIII or fragment thereof”, as used herein, is the product of a hybrid gene in which the coding sequence for one protein is extensively altered, for example, by fusing part of it to the coding sequence for a second protein from a different gene to produce a hybrid gene that encodes the fusion protein. As used herein, a fusion protein is a subset of the hybrid fVIII protein described in this application.

A “corresponding” nucleic acid or amino acid or sequence of either, as used herein, is one present at a site in a fVIII molecule or fragment thereof that has the same structure and/or function as a site in the fVIII molecule of another species, although the nucleic acid or amino acid number may not be identical. A DNA sequence “corresponding to” another fVIII sequence substantially corresponds to such sequence, and hybridizes to the sequence of the designated SEQ ID NO. under stringent conditions. A DNA sequence “corresponding to” another fVIII sequence also includes a sequence that results in the expression of a fVIII or fragment thereof and would hybridize to the designated SEQ ID NO. but for the redundancy of the genetic code.

A “unique” amino acid residue or sequence, as used herein, refers to an amino acid sequence or residue in the fVIII molecule of one species that is different from the homologous residue or sequence in the fVIII molecule of another species.

“Sequence homology” as used herein refers to identity or substantial similarity between two or more polypeptides or two or more nucleic acids. Sequence homology is determined on the basis of the nucleotide sequence of the two or more nucleic acids, or the amino acid sequence of the two or more polypeptides. The modified fVIII polypeptides of the present invention will have not more than 15%, usually not more than 5%, amino acid differences from the amino acid sequence recited in SEQ ID NO: 2, excluding the B domain.

“Specific activity,” as used herein, refers to the activity that will correct the coagulation defect of human fVIII deficient plasma. Specific activity is measured in units of clotting activity per milligram total fVIII protein in a standard assay in which the clotting time of human fVIII deficient plasma is compared to that of normal human plasma. One unit of fVIII activity is the activity present in one milliliter of normal human plasma. In the assay, the shorter the time for clot formation, the greater the activity of the fVIII being assayed. Porcine fVIII has coagulation activity in a human fVIII assay.

“Expression” refers to the set of processes that occur whereby genetic information is utilized to yield a product. For example, a DNA encoding the amino acid sequence of human fVIII having a number of amino acid substitutions can be “expressed” within a mammalian host cell to yield modified fVIII protein. The materials, genetic structures, host cells and conditions which permit expression of a given DNA sequence to occur are well-known in the art and can be manipulated to affect the time and amount of expression, as well as the intra- or extra-cellular location of the expressed protein. For example, by including DNA encoding a signal peptide at the 5′ end of the DNA encoding porcine fVIII (the 5′ end being, by convention, that end encoding the NH₂ terminus of the protein) the expressed protein becomes exported from the interior of the host cell into the culture medium. Providing a signal peptide coding DNA in combination with the modified fVIII coding DNA is advantageous because the expressed fVIII is exported into the culture medium which simplifies the process of purification. A preferred signal peptide is a mammalian fVIII signal peptide.

The human fVIII cDNA nucleotide sequence and predicted amino acid sequence are shown in SEQ ID NOs:1 and 2, respectively. FVIII is synthesized as an approximately 300 kDa single chain protein with internal sequence homology that defines the “domain” sequence NH₂-A1-A2-B-ap-A3-Cl-C2-COOH. In a fVIII molecule, a “domain”, as used herein, is a continuous sequence of amino acids that is defined by internal amino acid sequence identity and sites of proteolytic cleavage by thrombin. Unless otherwise specified, fVIII domains include the following amino acid residues, when the sequences are aligned with the human amino acid sequence (SEQ ID NO:2): A1, residues Ala1-Arg372; A2, residues Ser373-Arg740; B, residues Ser741-Arg1648; A3, residues Ser1690-Ile2032; C1, residues Arg2033-Asn2172; C2, residues Ser2173-Tyr2332. The remaining segment, residues Glu1649-Arg1689, is usually referred to as the fVIII light chain activation peptide. A “B-domainless” or “B domain deleted” (BDD) fVIII, or fragment thereof, as used herein, refers to a fVIII protein that lacks part or the entire B domain.

FVIII is proteolytically activated by thrombin or factor Xa, which dissociates it from von Willebrand factor, forming fVIIIa, which has procoagulant function. The biological function of fVIIIa is to increase the catalytic efficiency of factor IXa toward factor X activation by several orders of magnitude. Thrombin-activated fVIIIa is a 160 kDa A1/A2/A3-C1-C2 heterotrimer that forms a complex with factor IXa and factor X on the surface of platelets or monocytes. A “partial domain” as used herein is a continuous sequence of amino acids forming part of a domain.

“Subunits” of human or animal fVIII, as used herein, are the heavy and light chains of the protein. The A3-C1-C2 domains, residues Ser1690-Tyr2332, comprise the fVIII light chain. The A1-A2 domains, residues 1-740, make up the fVIII heavy chain.

The terms “epitope,” “antigenic site,” and “antigenic determinant,” as used herein, are used synonymously and are defined as a portion of the human, or animal fVIII, or fragments thereof, that is specifically recognized by an antibody. Lower antigenicity means antibodies, such as inhibitory antibodies, are less able to recognize or react to the site. An epitope (or antigenic site) can consist of any number of amino acid residues, and it can be dependent upon the primary, secondary, or tertiary structure of the protein.

The term “immunogenic site,” as used herein, is defined as a region of the human or animal fVIII, or fragments thereof, that specifically elicits the production of an antibody to the fVIII, or fragment, in a human or animal, as measured by routine protocols, such as immunoassay, e.g. ELISA, or the Bethesda assay, described herein. It can consist of any number of amino acid residues, and it can be dependent upon the primary, secondary, or tertiary structure of the protein. Lower immunogenicity means antibodies, such as inhibitory antibodies, are less likely to be produced by the host animal or human. In some embodiments, the modified or hybrid fVIII equivalent or fragment thereof is non-immunogenic (does not elicit the production of antibodies) or has lower immunogenicy in an animal or human than human fVIII.

As used herein, a “hybrid fVIII equivalent molecule or fragment thereof” or “hybrid equivalent fragment fVIII or fragment thereof” is an active fVIII, a hybrid fVIII molecule or fragment thereof comprising at least one sequence having one or more amino acid residues, which have no known sequence identity to human or animal fVIII sequences, substituted for at least one sequence having one or more specific amino acid residues in the human, animal, or hybrid fVIII or fragment thereof. The sequence of one or more amino acid residues that have no known identity to human or animal fVIII sequence is also referred to herein as “non-fVIII amino acid sequence”.

In an embodiment of the present invention, the amino acid(s), which are substituted into the fVIII and have no known sequence identity to human or animal fVIII, are alanine residues. In one embodiment, the specific fVIII sequence, for which the amino acid(s) having no known sequence identity to human or animal fVIII are substituted, includes an antigenic site that is immunoreactive with naturally occurring fVIII inhibitory antibodies, such that the resulting hybrid fVIII equivalent molecule or fragment thereof is less antigenic or not antigenic. In another embodiment, the specific fVIII sequence, for which the amino acid(s) having no known sequence identity to human or animal fVIII are substituted, includes an immunogenic site that elicits the formation of fVIII inhibitory antibodies in an animal or human, such that the resulting hybrid fVIII equivalent molecule or fragment thereof is less immunogenic or not immunogenic.

“fVIII deficiency,” as used herein, includes deficiency in clotting activity caused by production of defective fVIII, by inadequate or no production of fVIII, or by partial or total inhibition of fVIII by inhibitors. Hemophilia A is a type of fVIII deficiency resulting from a defect in an X-linked gene and the absence or deficiency of the fVIII protein it encodes.

As used herein, “diagnostic assays” include assays that in some manner utilize the antigen-antibody interaction to detect and/or quantify the amount of a particular antibody that is present in a test sample to assist in the selection of medical therapies. There are many such assays known to those of skill in the art. As used herein, human, animal or modified human fVIII DNA, or fragment thereof, and protein expressed therefrom, in whole or in part, can be substituted for the corresponding reagents in the otherwise known assays, whereby the modified assays may be used to detect and/or quantify antibodies to fVIII. It is the use of these reagents, the fVIII DNA, or fragment thereof, or protein expressed therefrom, that permits modification of known assays for detection of antibodies to human or animal fVIII. Such assays include, but are not limited to ELISAs, immunodiffusion assays, and immunoblots. Suitable methods for practicing any of these assays are known to those of skill in the art. As used herein, the fVIII or fragment thereof that includes at least one epitope of the protein can be used as the diagnostic reagent. Examples of other assays in which human, animal, such as porcine, or modified human fVIII or fragment thereof can be used include the Bethesda assay and anticoagulation assays.

The term “DNA encoding a protein, such as human fVIII” means a polydeoxynucleic acid whose nucleotide sequence embodies coding information to a host cell for the amino acid sequence of the protein, e.g. human fVIII, according to the known relationships of the genetic code.

The “expression product” of a DNA encoding a human or animal fVIII or a modified fVIII is the product obtained from expression of the referenced DNA in a suitable host cell, including such features of pre- or post-translational modification of protein encoded by the referenced DNA, including but not limited to glycosylation, proteolytic cleavage and the like. It is known in the art that such modifications can occur and can differ somewhat depending upon host cell type and other factors, and can result in molecular isoforms of the product, with retention of procoagulant activity. See, e.g. Lind, P. et al., Eur. J. Biochem. 232:1927 (1995), incorporated herein by reference.

An “expression vector” is a DNA element, often of circular structure, having the ability to replicate autonomously in a desired host cell, or to integrate into a host cell genome and also possessing certain well-known features which permit expression of a coding DNA inserted into the vector sequence at the proper site and in proper orientation. Such features can include, but are not limited to, one or more promoter sequences to direct transcription initiation of the coding DNA and other DNA elements such as enhancers, polyadenylation sites and the like, all as well known in the art. The term “expression vector” is used to denote both a vector having a DNA coding sequence to be expressed inserted within its sequence, and a vector having the requisite expression control elements so arranged with respect to an insertion site that it can serve to express any coding DNA inserted into the site, all as well-known in the art. Thus, for example, a vector lacking a promoter can become an expression vector by the insertion of a promoter combined with a coding DNA.

### General Description of Methods

The human fVIII gene was isolated and expressed in mammalian cells, as reported by Toole, J. J. et al. (1984) Nature 312:342-347; Gitschier, J. et al. (1984) Nature 312:326-330; Wood, W. I. et al. (1984) Nature 312:330-337; Vehar, G. A. et al. (1984) Nature 312:337-342; WO 87/04187; WO 88/08035; WO 88/03558; U.S. Pat. No. 4,757,006, and the amino acid sequence was deduced from cDNA. U.S. Pat. No. 4,965,199 to Capon et al. discloses a recombinant DNA method for producing fVIII in mammalian host cells and purification of human fVIII. Human fVIII expression on CHO (Chinese hamster ovary) cells and BHKC (baby hamster kidney cells) has been reported. Human fVIII has been modified to delete part or the entire B domain (U.S. Pat. No. 4,868,112), and replacement of the human fVIII B domain with the human factor V B domain has also been attempted (U.S. Pat. No. 5,004,803).

The cDNA sequence encoding human fVIII and predicted amino acid sequence are shown in SEQ ID NOs: 1 and 2, respectively. In SEQ ID NO: 1, the coding region begins at nucleotide position 208, the triplet GCC being the codon for amino acid number 1 (Ala) of the mature protein as given in SEQ ID NO: 2.

Human fVIII is isolated from plasma as a two subunit protein. The subunits, known as the heavy chain and light chain, are held together by a non-covalent bond that requires calcium or other divalent metal ions. The heavy chain of fVIII contains the A1 and A2 domains. The light chain of fVIII contains the A3, C1, and C2 domains. The B domain has no known biological function and can be removed, or partially removed, from the molecule proteolytically or by recombinant DNA technology methods without significant alteration in any measurable parameter of fVIII. Human recombinant fVIII has a similar structure and function to plasma-derived fVIII, though it is not glycosylated unless expressed in mammalian cells.

Human activated fVIII (“fVIIIa”) has three subunits due to cleavage of the heavy chain between the A1 and A2 domains. This structure is designated A1/A2/A3-C1-C2. Human fVIIIa is not stable under the conditions that stabilize porcine fVIIIa, presumably because of the weaker association of the A2 subunit of human fVIIIa. Dissociation of the A2 subunit of human and porcine fVIIIa is associated with loss of activity in the fVIIa molecule. Yakhyæv, A. et al., 1997, Blood 90:Suppl. 1, Abstract #126, reported binding of A2 domain by low density lipoprotein receptor-related protein, suggesting that cellular uptake of A2 mediated by such binding acts to down-regulate fVIII activity.

Previous U.S. patents have disclosed how to modify human fVIII with porcine fVIII. U.S. Pat. Nos. 5,663,060 and 5,364,771 describe hybrid human/animal, particularly human/porcine, fVIII molecules having coagulant activity, in which elements of the fVIII molecule of human or an animal are substituted for corresponding elements of the fVIII molecule of the other species. The cDNA sequence encoding the complete A2 domain of porcine fVIII, predicted amino acid sequence, and hybrid human/porcine fVIII having substitutions of all domains, all subunits, and specific amino acid sequences were disclosed in U.S. Pat. No. 5,364,771 and in WO 93/20093. More recently, the nucleotide and corresponding amino acid sequences of part of the A1 domain lacking the first 198 amino acid and of the A2 domain of porcine fVIII were reported in WO 94/11503, published May 26, 1994. The entire nucleotide sequence encoding porcine fVIII, including the complete A1 domain, activation peptide, A3, C1 and C2 domains, as well as the encoded amino acid sequence, was finally obtained by Lollar, as disclosed in U.S. Pat. No. 5,859,204, issued Jan. 12, 1999, and in WO 97/49725, published Dec. 31, 1997, both incorporated herein by reference. The amino acid sequence of porcine fVIII is given in SEQ ID NO: 3.

Since current information indicates that the B domain has no inhibitory epitope and has no known effect on fVIII function, in some embodiments the B domain is wholly or partially deleted in the active hybrid or hybrid equivalent fVIII molecules or fragments thereof prepared by any of the methods described herein. Expression of “B-domainless fVIII” is enhanced by including portions of the B-domain. The inclusion of the 3 amino acids of the B domain N-terminus and 11 amino acids of the B domain C-terminus was reported to result in favorable expression (Lind et al., 1995, Eur. J. Biochem. 232:19-27). “HBD” is a human fVIII that lacks the entire human B domain except for 5 amino acids of the B domain N-terminus and 9 amino acids of the B domain C-terminus (which results in the same 14 amino acid B domain linker sequence, S F S Q N P P V L K R H Q R, as disclosed in Lind et al.).

The purified modified fVIII or fragment thereof can be assayed for immunoreactivity and coagulation activity by standard assays including, for example, the plasma-free fVIII assay, the one-stage clotting assay, and the enzyme-linked immunosorbent assay using purified recombinant human fVIII as a standard.

Other vectors, including both plasmid and eukaryotic viral vectors, may be used to express a recombinant gene construct in eukaryotic cells depending on the preference and judgment of the skilled practitioner (see, for example, Sambrook et al., Chapter 16). Other vectors and expression systems, including bacterial, yeast, and insect cell systems, can be used but are not preferred due to differences in, or lack of, glycosylation.

Recombinant fVIII protein can be expressed in a variety of cells commonly used for culture and recombinant mammalian protein expression. In particular, a number of rodent cell lines have been found to be especially useful hosts for expression of large proteins. Preferred cell lines, available from the American Type Culture Collection, Rockville, Md., include baby hamster kidney cells, and Chinese hamster ovary (CHO) cells which are cultured using routine procedures and media.

FVIII Molecules with Reduced Immunoreactivity:

Epitopes that are immunoreactive with antibodies that inhibit the coagulant activity of fVIII (“inhibitors” or “inhibitory antibodies”) have been characterized based on known structure-function relationships in fVIII. Most inhibitory antibodies to human fVIII act by binding to epitopes located in the 40 kDa A2 domain or 20 kDa C2 domain of fVIII, disrupting specific functions associated with these domains, as described by Fulcher et al. (1985) Proc. Nat. Acad. Sci USA 82:7728-7732; and Scandella et al. (1988) Proc. Natl. Acad. Sci. USA 85:6152-6156. In addition to the A2 and C2 epitopes, there may be a third epitope in the A3 or C1 domain of the light chain of fVIII, according to Scandella et al. (1993) Blood 82:1767-1775. The significance of this putative third epitope is unknown, but it appears to account for a minor fraction of the epitope reactivity in fVIII.

After identification of clinically significant epitopes, recombinant fVIII molecules can be expressed that have less than or equal cross-reactivity compared with plasma-derived human or porcine fVIII when tested in vitro against a broad survey of inhibitor plasmas. Additional mutagenesis in epitopic regions can be done to reduce cross-reactivity. Reduced cross-reactivity, although desirable, is not necessary to produce a product that may have advantages over the existing plasma-derived human or porcine fVIII concentrate, which can produce side effects due to contaminant proteins or contaminant infectious agents such as viruses or prions. A recombinant fVIII or a modified recombinant fVIII molecule will not contain foreign proteins.

The basis for the greater coagulant activity of porcine fVIII appears to be the more rapid spontaneous dissociation of the human A2 subunit from human fVIIIa than the porcine A2 subunit from porcine fVIIIa. Dissociation of the A2 subunit leads to loss of activity, (Lollar, P. et al. (1990) J. Biol. Chem. 265:1688-1692; Lollar, P. et al. (1992) J. Biol. Chem. 267:23652-23657; Fay, P. J. et al. (1992) J. Biol. Chem. 267:13246-13250).

Anti-A2 antibodies block factor X activation, as shown by Lollar et al. (1994) J. Clin. Invest. 93:2497-2504. Previous mapping studies by deletion mutagenesis described by Ware et al. (1992) Blood Coagul. Fibrinolysis 3:703-716, located the A2 epitope to within a 20 kDa region of the NH₂-terminal end of the 40 kDa A2 domain. Competition immunoradiometric assays have indicated that A2 inhibitors recognize either a common epitope or narrowly clustered epitopes, as described by Scandella et al. (1992) Throm. Haemostas. 67:665-671, and as demonstrated in U.S. Pat. No. 5,859,204.

Hemophilia A inhibitor patients and patients with acquired hemophilia A recognize immunodominant epitopes in the A2 and C2 domains of human fVIII. Hemophilia A mice also recognize A2 and C2 domain epitopes when immunized with human fVIII using a dosing schedule that mimics use in human hemophilia A. The immune response of hemophilia A mice to human and procine fVIII was compared using a domain specific ELISA. In this assay, monoclonal antibodies were tested against a panel of six single human fVIII domain hybrid human/porcine fVIII molecules as antigens that contain the human A1, A2, ap, A3, C1 or C2 domains. With anti-human antibodies, a positive signal with one of the single human domain proteins identifies domain specificity, whereas loss of signal indicates domain specificity of anti-porcine fVIII antibodies. Exon 16 (E16)-disrupted hemophilia A mice (n=3) received six weekly 10 μg/kg intravenous injections of recombinant B-domain deleted human fVIII and a final 25 μg/kg boost. To obtain comparable inhibitor titers, E16 mice (n=3) received six weekly injections of 40 μg/kg of recombinant B-domain deleted porcine fVIII. Spleens from high titer mice were fused with NS1 mouse myeloma cells and 485 of the resulting hybridomas were analyzed (Table 1).

TABLE 1
Domain Specificity

| Mouse Immunogen | | | | | | | | | | |
|---|---|---|---|---|---|---|---|---|---|---|
| Hybridomas | | | A1 | A2 | ap | A3 | C1 | C2 | CR | MD |
| A | Human fVIII | 95 | 2 | 16 | 0 | 2 | 7 | 21 | 23 | 24 |
| B | Human fVIII | 126 | 13 | 23 | 0 | 1 | 2 | 27 | 39 | 21 |
| C | Human fVIII | 54 | 1 | 15 | 1 | 2 | 1 | 10 | 9 | 15 |
| D | Porcine fVIII | 123 | 39 | 7 | 1 | 19 | 8 | 16 | 33 | 0 |
| E | Porcine fVIII | 27 | 13 | 5 | 0 | 0 | 0 | 4 | 2 | 3 |
| F | Porcine fVIII | 60 | 9 | 6 | 0 | 12 | 1 | 9 | 13 | 10 |

CR: Cross-reactive
MD: Multidomain

Human fVIII elicited a significantly greater number of antibodies to the A2 domain, whereas porcine fVIII elicited a significantly greater number of antibodies to the A1 and A3 domains (p<0.01, chi square test). The greater number of anti-C2 antibodies to human fVIII was not statistically significant (p=0.10) in this experiment. The differential immunodominance of human and porcine fVIII epitopes suggests that it may be possible to design a recombinant hybrid human/porcine fVIII molecule that is less immunogenic than human fVIII in the treatment of patients with hemophilia A.

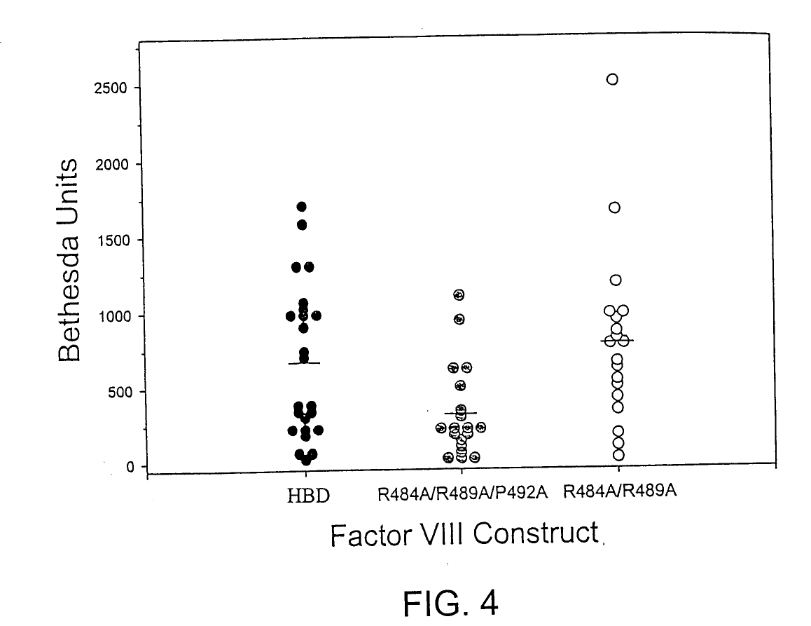

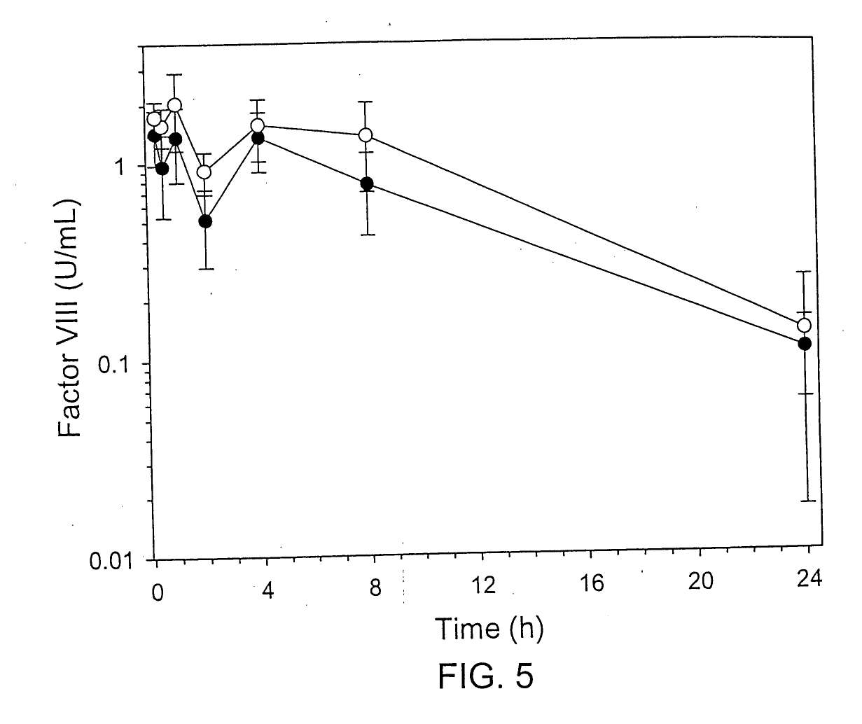

A triple mutant, R484A/R489A/P492A, which substitutes three alanine residues in the A2 epitope of a human B domain deleted fVIII (HBD), was found to be significantly less immunogenic than the unsubstituted B domain deleted human fVIII in hemophilia A mice (FIG. 4), while retaining full procoagulant activity. In contrast, the double mutant R484A/R489A, which substitutes two alanine residues in a B domain deleted human fVIII, was not less immunogenic than HBD. The clearance and hemostatic efficacy of R484A/R489A/P492A was similar to HBD (FIG. 5 and Table 2), making it unlikely the reduced immunogenicity of R484A/R489A/P492A was due to decreased bioavailability.

The decreased immunogenicity of R484A/R489A/P492A was identified by a reduction in the Bethesda inhibitor titer. In addition to inhibitory anti-A2 antibodies, the Bethesda assay can detect loss of fVIII coagulant function due to anti-C2 and other antibodies. In patient plasmas, anti-A2 and anti-C2 antibodies appear to contribute similarly to the inhibitor titer. In a previous study of 34 hemophilia A inhibitor patient plasmas, inhibitor titers were reduced by an average of 40% and 30% by soluble recombinant A2 and C2 domains, respectively (Prescott et al. (1997) Blood 89:3663-3671). The results using the R484A/R489A/P492A mutant were consistent with this previous study in that mutagenesis of A2 epitope resulted in a partial reduction of the inhibitor titer. Preliminary characterizations by homolog scanning mutagenesis of the B cell epitopes recognized by inhibitory antibodies in the murine model indicates that the A2 and C2 domains are the most frequently targeted domains as they are in humans. This result establishes the mouse model as a reliable predictive model of human efficacy. A combination of mutations in the A2 and C2 domains could further reduce the immunogenicity of human fVIII.

In contrast to the Bethesda assays, there was no significant difference among the treatment groups by anti-fVIII ELISA, which detects both inhibitory and non-inhibitory antibodies. Non-inhibitory antibodies and anti-fVIII antibodies have been identified in human hemophilia A patients (Giles et al., (1993) Blood 82:2452-2461). The relative contribution of inhibitory antibodies to the total ELISA signal is not known in human or murine hemophilia A. However, the results of the present invention indicate that the ELISA assay is not sensitive to the reduction of immunogenicity of inhibitor epitopes because non-inhibitory antibodies also contribute to the total ELISA signal.

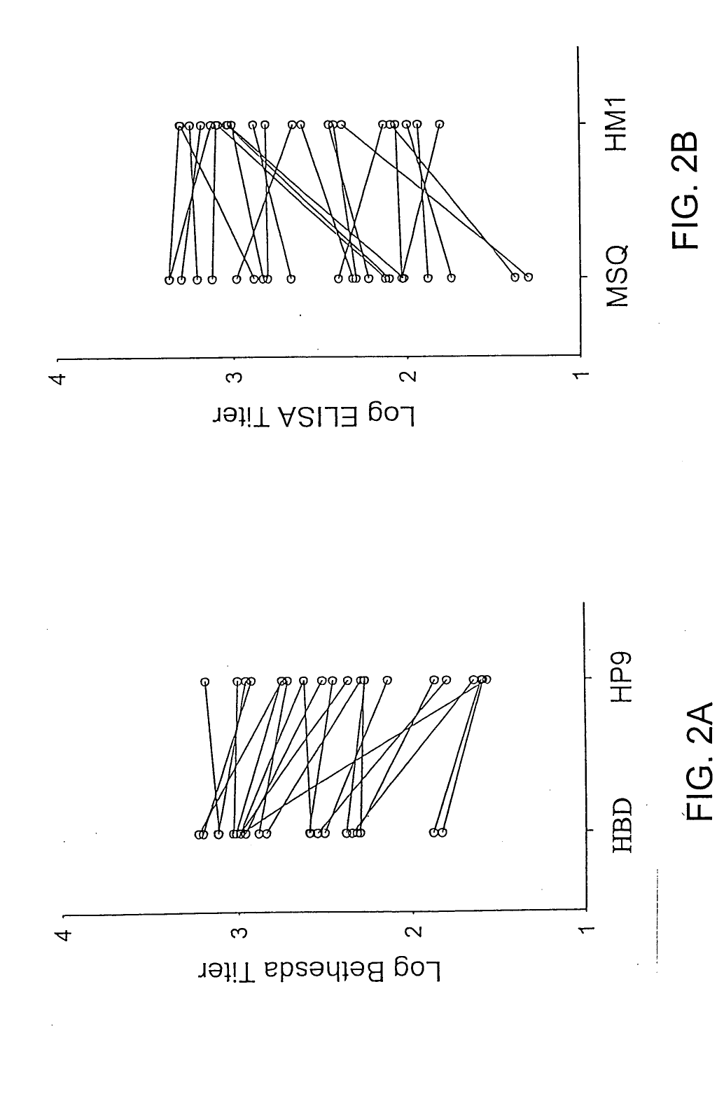

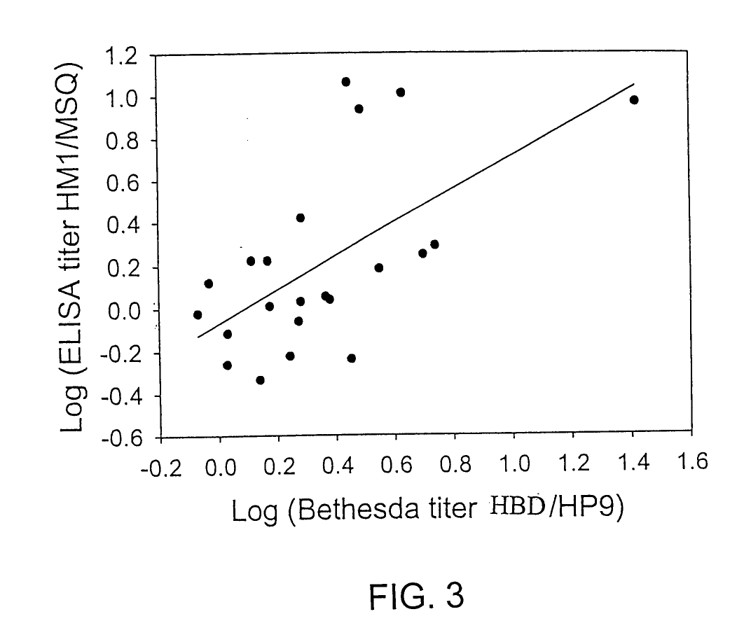

The immunodominance of the A2 epitope in hemophilia A mice was tested in plasmas from mice immunized with human B domainless fVIII using hybrid human/porcine and human/murine fVIII constructs. HP9, which is human except for insertion of porcine sequence within the 484-508 segment of the A2 domain, was less antigenic than HBD (FIG. 2A). In contrast, HM1, which is murine except for insertion of human sequence within the 484-508 segment, is more antigenic than murine B domainless fVIII (MBD) (FIG. 2B). Furthermore, there is a correlation between the reduction in antigenicity of HP9 compared to HBD and the increase in antigenicity of HM1 compared to MBD (FIG. 3) indicating that the antigenicity of the A2 epitope in individual mice is detected similarly in both assay systems.

A2 and C2 domain epitopes are immunodominant in fVIII inhibitor patients regardless of whether antibodies arise in the disparate immunological settings of alloimmunity or autoimmunity (Prescott et al., (1997) Blood 89:3663-3671). The R484-I508 A2 segment appears to encompass the only inhibitory A2 epitope recognized by most patients (Healey et al., (1995) J. Biol. Chem. 270:14505-14509). This segment is predicted to consist of a large, surface exposed loop based on the homology model of the fVIII A domains (Pemberton et al., (1997) Blood 89:2413-2421). Inhibitory anti-C2 antibodies recognize a discontinuous epitope that contains a functionally important phospholipid binding site (Barrow et al., (2001) Blood 97:169-174; Arai et al., (1989) J. Clin. Invest. 83:1978-1984; Pratt et al., (1999) Nature 402:439-442; Spiegel et al., (2001) Blood 98:13-19). Although epitope-specific frequency distributions of the antibody populations in fVIII inhibitor patients or hemophilia A mice have not been enumerated, the B cell response to fVIII appears more restricted that the T cell response. For example, antibodies to the A1 domain are rarely seen, whereas the population of fVIII-specific T cells recognizes all of the domains in the human (Reding et al., (2000) Thromb. Haemost. 84:643-652) and murine (Wu et al., (2001)) Thromb. Haemost. 85:125-133) inhibitor response. Additionally, in contrast to the presence of common immunodominant B cell epitopes, immunodominant T cell epitopes have not been identified.

It has been established herein that structural modification of the fVIII molecule can reduce the immunogenicity in a murine hemophilia A model. The murine model can guide preclinical development of a therapeutically useful low immunogenic form of fVIII for human therapy. In one embodiment of the present invention, a method of identifying a modified fVIII having reduced immunogenicity or antigenicity is provided. This method comprises the steps: injecting at least one dose of said modified fVIII into a first group of animal (such as mice) test subjects; injecting at least one dose of an unmodified fVIII or a fVIII with known antigenic and/or immunogenic properties into a second group of animal test subjects; and using a diagnostic assay to compare the inhibitory antibodies produced by said first group of animal test subjects with inhibitory antibodies produced by said second group of animal test subjects.

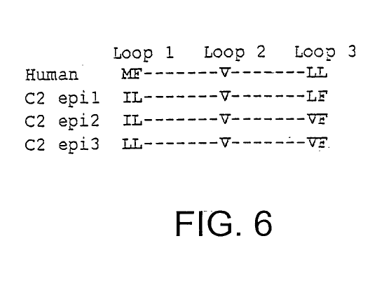

Three human B domain deleted fVIII constructs, A2C2epi1, A2C2epi2 and A2C2epi3, contain the R484A/R489A/P492 mutation in the A2 domain and additional C2 domain mutations. In an attempt to conserve function, amino acids were selected for replacement of human residues at loops 1 and 3 (FIG. 6) in the C2 domain based on the sequences of other species for which the fVIII is known (Table 3). These three constructs were compared with human B domain deleted fVIII (HBD), R484A/R489A/P492A (A2epi7), and each other.

A2C2epi1 contained the additional mutations M2199I/F2200L/L2252F in the C2 domain.

A2C2epi2 contained the additional mutations M2199I/F2200L/L2251V/L2252F in the C2 domain.

A2C2epi3 contained the additional mutations M2199L/F2200L/L2251V/L2252F in the C2 domain. A2C2epi3 differs from A2C2epi2 in that A2C2epi3 substitutes leucine for M2199 instead of isoleucine.

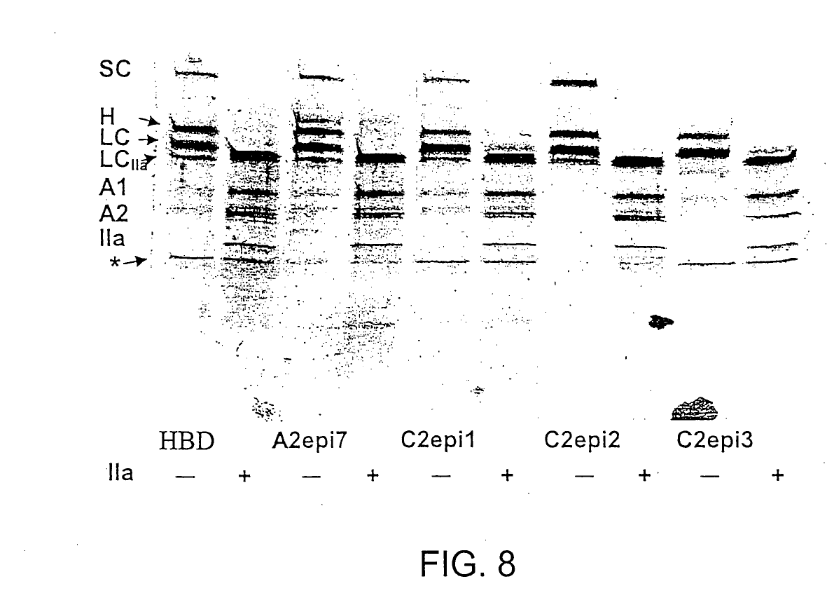

The five constructs, HBD, A2epi7, A2C2epi1, A2C2epi2 and A2C2epi3, were expressed in BHK-derived cells and purified. The specific activities of A2C2epi1, A2C2epi2 and C3epi3 were similar to HBD (Table 4) and the purity of all the preparations was considered acceptable (FIG. 8).

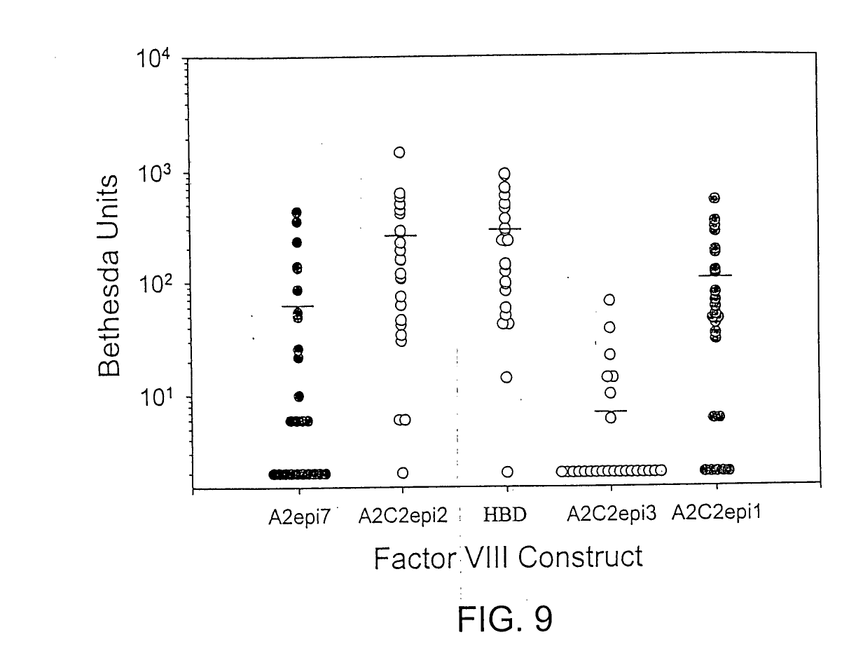

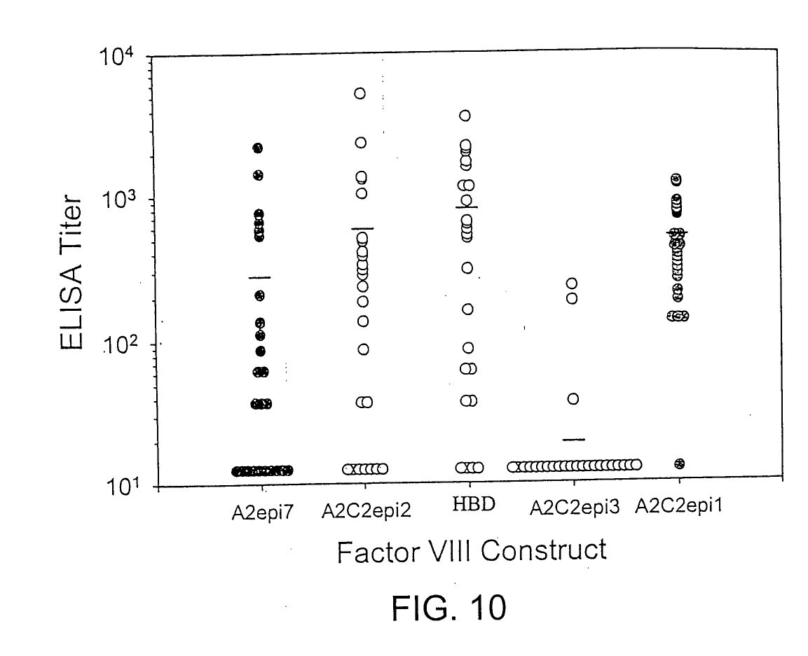

A2C2epi3 was less immunogenic than HBD and A2epi7 (FIGS. 9 and 10). In the Bethesda assay, A2C2epi1 was less immunogenic than HBD but not A2epi7. A2C2epi2 was not significantly less immunogenic than HBD in either assay, even though it contains the low immunogenicity R484A/R489A/P492A mutation. Conceivably, the C2 domain of A2C2epi2 is more immunogenic than the HBD, with the C2 domain offsetting a reduction of the immunogenicity of the A2 domain.

Diagnostic Assays

The fVIII cDNA and/or protein expressed therefrom, in whole or in part, can be used in assays as diagnostic reagents for the detection of inhibitory antibodies to human or animal fVIII or modified animal VIII in substrates, including, for example, samples of serum and body fluids of human patients with fVIII deficiency. These antibody assays include assays such as ELISA assays, immunoblots, radioimmunoassays, immunodiffusion assays, and assay of fVIII biological activity (e.g., by coagulation assay). Techniques for preparing these reagents and methods for use thereof are known to those skilled in the art.

Nucleic acid and amino acid probes can be prepared based on the sequence of the modified fVIII cDNA or protein molecule or fragments thereof. In some embodiments, these can be labeled using dyes or enzymatic, fluorescent, chemiluminescent, or radioactive labels that are commercially available. The amino acid probes can be used, for example, to screen sera or other body fluids where the presence of inhibitors to human, animal, or hybrid human/animal fVIII is suspected. Levels of inhibitors can be quantitated in patients and compared to healthy controls, and can be used, for example, to determine whether a patient with a fVIII deficiency can be treated with an animal or modified animal fVIII. The cDNA probes can be used, for example, for research purposes in screening DNA libraries.

Preparation of Recombinant FVIII

Recombinant fVIII can be produced through the use of eukaryotic protein expression systems. In general, a eukaryotic cell line, which is deficient in a required gene, is transformed with a vector comprising the gene that it has a deficiency for, and the recombinant DNA which one wishes to express. Transformation can be accomplished by techniques such as electroporation or viral delivery. The cell line chosen to produce the protein is selected to be compatible with the protein of interest, capable of continuously expressing the protein of interests, capable of growing on a medium, which facilitates purification of the protein of interest, along with other factors known to those skilled in the art. Examples of such techniques are disclosed in European Patent Application 0 302 968 A2 and U.S. Pat. No. 5,149,637 both of which are incorporated by reference in their entirety.

Testing of Recombinant FVIII Molecules

The recombinant fVIII molecules can be tested in humans for their reduced antigenicity and/or immunogenicity in at least two types of clinical trials. In one type of trial, designed to determine whether the recombinant or recombinant hybrid fVIII is antigenic with inhibitory antibodies, recombinant fVIII or recombinant modified fVIII is administered, preferably by intravenous infusion, to approximately 25 patients having fVIII deficiency who have antibodies to fVIII that inhibit the coagulant activity of therapeutic human or porcine fVIII. The dosage of the recombinant or recombinant modified fVIII is in a range between 5 and 50 Units/kg body weight, preferably 10-50 Units/kg, and most preferably 40 Units/kg body weight. Approximately 1 hour after each administration, the recovery of fVIII from blood samples is measured in a one-stage coagulation assay. Samples are taken again approximately 5 hours after infusion, and recovery is measured. Total recovery and the rate of disappearance of fVIII from the samples are predictive of the antibody titer and inhibitory activity. If the antibody titer is high, fVIII recovery usually cannot be measured. The recovery results are compared to the recovery results in patients treated with plasma-derived human fVIII, recombinant human fVIII, porcine fVIII, and other commonly used therapeutic forms of fVIII or fVIII substitutes.

In a second type of clinical trial, designed to determine whether the recombinant or recombinant modified fVIII is immunogenic, i.e., whether patients will develop inhibitory antibodies, recombinant or recombinant hybrid fVIII is administered, as described in the preceding paragraph, to approximately 100 previously untreated hemophiliac patients who have not developed antibodies to fVIII. Treatments are given approximately every 2 weeks over a period of 6 months to 1 year. At 1 to 3 month intervals during this period, blood samples are drawn and Bethesda assays or other antibody assays are performed to determine the presence of inhibitory antibodies. Recovery assays can also be done, as described above, after each infusion. Results are compared to hemophiliac patients who receive plasma-derived human fVIII, recombinant human fVIII, porcine fVIII, or other commonly used therapeutic forms of fVIII or fVIII substitutes.

Pharmaceutical Compositions

Pharmaceutical compositions comprising recombinant or recombinant modified fVIII, alone or in combination with appropriate pharmaceutical stabilization compounds, delivery vehicles, and/or carrier vehicles, are prepared according to known methods, as described in Remington's Pharmaceutical Sciences by E. W. Martin.

In one preferred embodiment, the preferred carriers or delivery vehicles for intravenous infusion are physiological saline or phosphate buffered saline.

In another preferred embodiment, suitable stabilization compounds, delivery vehicles, and carrier vehicles include but are not limited to other human or animal proteins such as albumin.

Phospholipid vesicles or liposomal suspensions are also preferred as pharmaceutically acceptable carriers or delivery vehicles. These can be prepared according to methods known to those skilled in the art and can contain, for example, phosphatidylserine/phosphatidylcholine or other compositions of phospholipids or detergents that together impart a negative charge to the surface, since fVIII binds to negatively charged phospholipid membranes. Liposomes may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the hybrid or modified fVIII is then introduced into the container. The container is then swirled by hand to free lipid material from the sides of the container and to disperse lipid aggregates, thereby forming the liposomal suspension.

Recombinant or recombinant modified FVIII can be combined with other suitable stabilization compounds, delivery vehicles, and/or carrier vehicles, including vitamin K dependent clotting factors, tissue factor, and von Willebrand factor (vWf) or a fragment of vWf that contains the fVIII binding site, and polysaccharides such as sucrose.

Recombinant or recombinant modified fVIII can also be delivered by gene therapy in the same way that human fVIII can be delivered, using delivery means such as retroviral vectors. This method consists of incorporation of fVIII cDNA into human cells that are transplanted directly into a fVIII deficient patient or that are placed in an implantable device, permeable to the fVIII molecules but impermeable to cells, which are then transplanted. The preferred method will be retroviral-mediated gene transfer. In this method, an exogenous gene (e.g., a fVIII cDNA) is cloned into the genome of a modified retrovirus. The gene is inserted into the genome of the host cell by viral machinery where it will be expressed by the cell. The retroviral vector is modified so that it will not produce virus, preventing viral infection of the host. The general principles for this type of therapy are known to those skilled in the art and have been reviewed in the literature (e.g., Kohn, D. B. et al. (1989) Transfusion 29:812-820).

Recombinant or recombinant modified fVIII can be stored bound to vWf to increase the half-life and shelf-life of the modified molecule. Additionally, lyophilization of fVIII can improve the yields of active molecules in the presence of vWf. Current methods for storage of human and animal fVIII used by commercial suppliers can be employed for storage of hybrid fVIII. These methods include: (1) lyophilization of fVIII in a partially-purified state (as a fVIII “concentrate” that is infused without further purification); (2) immunoaffinity-purification of fVIII by the Zimmerman method and lyophilization in the presence of albumin, which stabilizes the fVIII; (3) lyophilization of recombinant fVIII in the presence of albumin.

Additionally, hybrid or modified fVIII has been indefinitely stable at 4° C. in 0.6 M NaCl, 20 mM MES, and 5 mM CaCl₂ at pH 6.0 and also can be stored frozen in these buffers and thawed with minimal loss of activity.

Methods of Treatment

Recombinant or recombinant modified fVIII is used to treat uncontrolled bleeding due to fVIII deficiency (e.g., intraarticular, intracranial, or gastrointestinal hemorrhage) in hemophiliacs with and without inhibitory antibodies and in patients with acquired fVIII deficiency due to the development of inhibitory antibodies. The active materials are preferably administered intravenously. It is especially useful to treat naïve patients, i.e., patients who have not yet developed inhibitory antibodies, with FVIII molecules having low immunogenicity in order to reduce the likelihood of the patient developing inhibitory antibodies.

Additionally, recombinant or recombinant modified fVIII can be administered by transplant of cells genetically engineered to produce the hybrid or by implantation of a device containing such cells, as described above.

In a preferred embodiment, pharmaceutical compositions of recombinant or recombinant modified fVIII alone or in combination with stabilizers, delivery vehicles, and/or carriers are infused into patients intravenously according to the same procedure that is used for infusion of human or animal fVIII.

The treatment dosages of recombinant or recombinant modified fVIII composition that must be administered to a patient in need of such treatment will vary depending on the severity of the fVIII deficiency. Generally, dosage level is adjusted in frequency, duration, and units in keeping with the severity and duration of each patient's bleeding episode. Accordingly, the modified fVIII is included in the pharmaceutically acceptable carrier, delivery vehicle, or stabilizer in an amount sufficient to deliver to a patient a therapeutically effective amount of the fVIII to stop bleeding, as measured by standard clotting assays.

FVIII is classically defined as that substance present in normal blood plasma that corrects the clotting defect in plasma derived from individuals with hemophilia A. The coagulant activity in vitro of purified and partially-purified forms of fVIII is used to calculate the dose of fVIII for infusions in human patients and is a reliable indicator of activity recovered from patient plasma and of correction of the in vivo bleeding defect. There are no reported discrepancies between standard assay of novel fVIII molecules in vitro and their behavior in the dog infusion model or in human patients, according to: Lusher, J. M. et al. 328 New Engl. J. Med. 328:453-459; Pittman, D. D. et al., (1992) Blood 79:389-397; and Brinkhous et al. (1985) Proc. Natl. Acad. Sci. 82:8752-8755.

Usually, the desired plasma fVIII level to be achieved in the patient through administration of the recombinant or recombinant modified fVIII is in the range of 30-100% of normal. In a preferred mode of administration of the recombinant or recombinant modified fVIII, the composition is given intravenously at a preferred dosage in the range from about 5 to 50 units/kg body weight, more preferably in a range of 10-50 units/kg body weight, and most preferably at a dosage of 20-40 units/kg body weight; the interval frequency is in the range from about 8 to 24 hours (in severely affected hemophiliacs); and the duration of treatment in days is in the range from 1 to 10 days or until the bleeding episode is resolved. See, e.g., Roberts, H. R., and M. R. Jones, Ch. 153, 1453-1474, 1460, in Hematology, Williams, W. J., et al., ed. (1990). Patients with inhibitors may require more recombinant or recombinant modified fVIII, or patients may require less recombinant or recombinant modified fVIII because of its higher specific activity than human fVIII or decreased antibody reactivity or immunogenicity. As in treatment with human or porcine fVIII, the amount of recombinant or recombinant modified fVIII infused is defined by the one-stage fVIII coagulation assay and, in selected instances, in vivo recovery is determined by measuring the fVIII in the patient's plasma after infusion. It is to be understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

Treatment can take the form of a single intravenous administration of the composition or periodic or continuous administration over an extended period of time, as required. Alternatively, recombinant or recombinant modified fVIII can be administered subcutaneously or orally with liposomes in one or several doses at varying intervals of time.

FVIII can also be used to treat uncontrolled bleeding due to fVIII deficiency in hemophiliacs who have developed antibodies to human fVIII. In this case, coagulant activity that is superior to that of human or animal fVIII alone is not necessary. Coagulant activity that is inferior to that of human fVIII (i.e., less than 3,000 units/mg) will be useful if that activity is not neutralized by antibodies in the patient's plasma.

The recombinant or recombinant modified fVIII molecule and the methods for isolation, characterization, making, and using it generally described above will be further understood with reference to the following non-limiting examples.

### EXAMPLES

### Example 1

### Construction and Evaluation of the R484A/R489A/P492A (A2epi7) Mutant

Materials—Citrated hemophilia A plasma and normal pooled human plasma (FACT) were purchased from George King Biomedical, Inc. (Overland Park, Kans.). Activated partial thromboplastin time reagent (Automated APTT®) was purchased from Biomerieux (Durham, N.C.). Murine anti-human fVIII monoclonal antibodies ESH4, ESH5 and ESH8 were purchased from American Diagnostica. Synthetic oligonucleotides were purchased from Life Technologies. Restriction enzymes were purchased from New England Biolabs or Promega. A cell line derived from baby hamster kidney cells was a generous gift from Dr. R. T. A. Macgillivray (Funk et al., 1990, Biochemistry 29:1654-1660.). Exon 16-disrupted (E16) hemophilia A mice in a C57BL/6 background were obtained from Dr. Leon Hoyer and a breeding colony was established (Bi et al., 1995, Nat. Genet. 10:119-121). Nine- to twelve-week old E16 male or female hem A or normal C57BL/6 mice were used in the experiments. Novel fVIII DNA sequences generated by PCR were confirmed by dideoxy sequencing using an Applied Biosystem 373a automated DNA sequencer and the PRISM dye terminator kit.

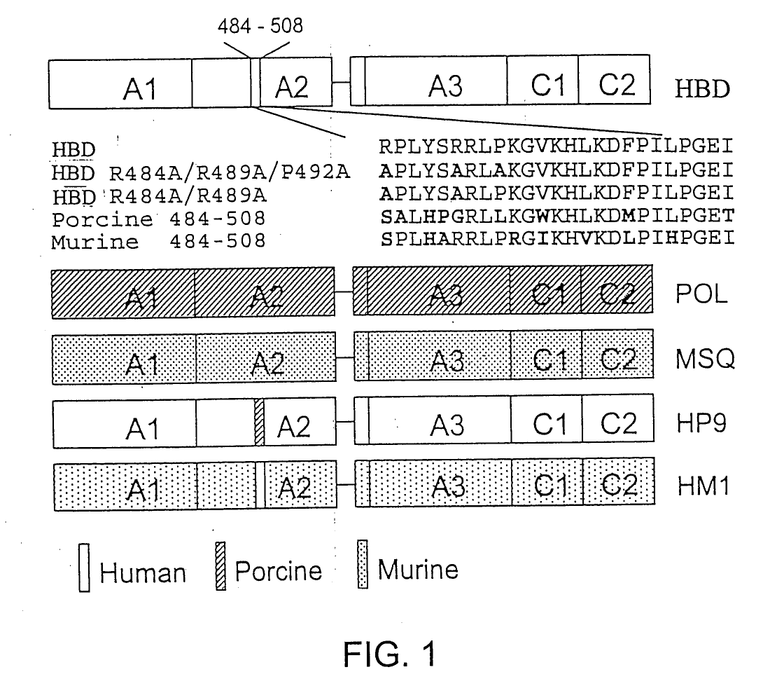

Construction of recombinant fVIII mutant cDNAs—The cDNA encoding a human B-domain deleted (HBD) form of fVIII was prepared as described in Doering et al., 2002, J. Biol. Chem. 277:38345-38349. It contains a S F S Q N P P V L K R H Q R linker sequence between the A2 and ap-A3 domains. The linker corresponds to the first five and last nine amino acids of the B domain and contains a recognition sequence for intracellular PACE/furin processing. This produces A1-A2/ap-A3-C1-C2 heterodimeric fVIII as the dominant secreted species, which is considered the physiologic form. The cDNAs for porcine and murine B-domain deleted forms of fVIII, designated PBD and MBD, respectively, which also contain PACE/furin recognition linker sequences, were prepared as described previously (Doering et al., 2002, J. Biol. Chem. 277:38345-38349; Doering et al., 2002, Thromb. Haemost. 88:450-458). The cDNA for a B domainless hybrid/porcine fVIII molecule designated HP9, which contains insertion of the porcine segment corresponding to residues 484-508 in the A2 domain of human fVIII (FIG. 1), has been described previously (Healey et al., 1995, J. Biol. Chem. 270:14505-14509). A B domainless hybrid human/murine fVIII cDNA, HM1, encoding a fVIII molecule that is murine except for a human segment corresponding to residues 484-508 of the A2 domain (FIG. 1), was constructed by splicing-by-overlap extension mutagenesis using MBD as the template.

The construction of a cDNA encoding a B domainless R489A human fVIII has been described previously (Lubin et al., 1997, J. Biol. Chem. 272:30191-30195) and was modified further by insertion of DNA encoding the S F S Q N P P V L K R H Q R linker sequence. The resulting cDNA was used as a template for the production cDNAs encoding R484A/R489A and R484A/R489A/P492A human fVIII (FIG. 1) by splicing-by-overlap extension mutagenesis. For the R484A/R489A mutant, 5′-CAC GGA ATC ACT GAT GTC GCC CCT TTG TAT TCA GCC AGA-3′ and 5′-TCT GGC TGA ATA CM AGG GGC GAC ATC AGT GAT TCC GTG-3′ were used as the mutagenic sense and antisense primers, respectively. For the R484A/R489A/P492A mutant 5′-ACT GAT GTC GCC CCT TTG TAT TCA GCC AGA TTA GCC AAA-3′ and 5′-TTT GGC TAA TCT GGC TGA ATA CM AGG GGC GAC ATC AGT-3′, were used as the mutagenic sense and antisense primers, respectively.

Expression and purification of recombinant fVIII molecules—Recombinant fVIII molecules were expressed in baby hamster kidney-derived cells in serum-free medium using the ReNeo expression vector as described previously in Healey et al., 1998, Blood 92:3701-3709. HBD, R484A/R489A, R484A/R489A/P492A, MBD, PBD and HM1 were purified by SP-Sepharose Fast Flow and Source Q or Mono Q ion-exchange chromatography essentially as describe previously for HBD, MBD and PBD in Doering et al., 2002, J. Biol. Chem. 277:38345-38349; and Doering et al., 2002, Thromb. Haemost. 88:450-458.

HP9 was purified using the following procedure. Ammonium sulfate was added to 5.9 liters of cell culture medium at 4° C. to 65% saturation and allowed to stir overnight. The precipitate was collected by centrifugation, dialyzed against 0.15 M NaCl, 0.02 M Hepes, 5 mM CaCl₂, 0.01% Tween-80, pH 7.4, and applied to a 1.5×10 cm ESH5—Sepharose column equilibrated in the same buffer. HP9 was eluted with 1 M NaCl, 5 mM Mes, 2.5 mM CaCl₂, 50% ethylene glycol (v/v), pH 6.0. Fractions from the fVIII activity peak were diluted 1/5 into 0.04 M Hepes, 5 mM CaCl₂, 0.01% Tween-80, pH 7.4 and further purified using Mono Q ion-exchange chromatography.

Purified proteins were at least 90% pure as judged by sodium dodecyl sulfate-polyacrylamide gel electrophoresis and contained heterodimeric fVIII as the dominant species. Concentrations were calculated using the absorbance at 280 nm of the purified proteins and molar extinction coefficients that were estimated from the respective deduced amino acid compositions (Pace et al., 1995, Protein Sci. 4:2411-2423). The specific activities of the purified proteins were calculated using the protein concentration and fVIII coagulant activity, which was measured as described below against a human fVIII plasma standard. The following specific activities were obtained: HBD, 1300 U/nmole; R484A/R489A/P492A, 1270 U/mole; R484A/R489A/P492A, 1460 U/nmol; HP9 1100, U/nmol; HM1 540, U/nmol. The specific activities of the PBD and MBD preparations have been previously reported as 2050 U/nmol and 660 U/nmol, respectively (Doering et al., 2002, J. Biol. Chem. 277:38345-38349; and Doering et al., 2002, Thromb. Haemost. 88:450-458). The deduced polypeptide chain molecular mass for all fVIII species used in this study is 165 kDa and was for conversion of fVIII concentrations to mass units.

Immunization of hemophilia A mice with HBD, R484A/R489A/P492A and R484A/R489A—Preparations of HBD, R484A/R489A/P492A and R484A/R489A were diluted to 0.17 mg/ml in 0.4 M NaCl, 20 mM HEPES, 5 mM CaCl₂0.01% Tween-80 pH 7.4 and stored in small aliquots at −80° C. Samples were diluted were diluted to 4 μg/ml in sterile normal saline immediately prior to injection. Eighty-three hemophilia A mice were divided into HBD (n=24), R484A/R489A/P492A (n=23), R484A/R489A (n=24), buffer injected control (n=5) and non-injected control (n=7) groups. Mice were warmed under a 75-watt lamp for 3 to 4 minutes to dilate tail veins before injection. Silver nitrate was used to cauterize bleeding after injections and tail snips. Recipients of different test materials were mixed within each cage and remained in their original cages to avoid fighting. Approximately equal numbers of males and females were in each group. Mice received six injections of 10 μg/kg body weight (˜80 U/kg) at 14 day intervals, followed by a final rejection of 25 μg/kg body weight (˜200 U/kg) two weeks after the sixth dose. Blood was collected into 1/10 volume 3.8% trisodium citrate by tail snip under metofane anesthesia 13 days after the fourth injection and by terminal cardiac puncture 13-14 days after the final injection. Samples were held on ice prior to centrifugation at 3000×g for 15 minutes at 4° C. to collect plasma. During the course of the experiment 0, 4, 5, 1 and 2 mice died in the HBD, R484A/R489A/P492A, R484A/R489A, buffer injected control and non-injected control groups, respectively. The overall mortality during this experiment was 14%, which was considered acceptable given the increased mortality of hemophilia A mice associated with handling (Bi et al., 1995, Nat. Genet. 10:119-121).

In vivo clearance of HBD and R484A/R489A/P492A—For clearance studies, HBD or R484A/R489A/P492A were diluted to 50 U/ml (−6.25 μg/ml) in sterile saline for injection immediately before use. Hemophilia A mice were anesthetized with metofane, weighed, warmed under a 75-watt lamp for 3 minutes to dilate veins and injected with 100 U/kg (˜12.5 μg/kg) by tail vein. Silver nitrate was used to cauterize the injection sites. At various time, mice were anesthetized by intraperitoneal injection of a mixture of 300 mg/kg ketamine and 75 mg/kg xylazine and blood was collected by cardiac puncture into 1/10 volume of 3.8% trisodium citrate. Because serial sampling from hemophilia A mice is difficult, three mice were used for each time point for each fVIII construct. Plasma samples were prepared by centrifugation and stored at −70° C. FVIII activity was determined by chromogenic assay as described below.

Hemostatic efficacy of HBD and R484A/R489A/P492A—The hemostatic efficacy of R484A/R489A/P492A in hemophilia A mice was measured a using a tail vein transection model as described previously (Parker et al., 2003, Thromb. Haemost. 89:480-485). Briefly, mice were anesthetized with 1.5 mg/kg droperidol/75 mg/kg ketamine intraperitoneally, warmed to dilate the tail veins, and injected with R484A/R489A/P492A. After anesthesia was deepened using methoxyflurane, mice were placed in a 50 ml conical restraint tube, the distal 1 cm of tail was transected and the stump was placed in a test tube containing 150 mM NaCl at 37° C. At 2 h, surviving mice were caged and mortality at 24 h was determined. The up-and-down method for small samples (Dixon, W. J., 1965, J. Amer. Stat. Assoc. 60:967-978; and Dixon and Massey, 1969, in Introduction to Statistical Analysis, McGraw Hill, New York, 377-394) was used to estimate the dose that produces 50% survival (ED₅₀). An initial dose of fVIII was given to a single mouse as a priori estimate of the ED₅₀. If the mouse survived the 24 h test period, another mouse was tested and the dose was decreased. If the subject died, the dose was increased in the next subject. Testing was continued until a chosen nominal sample size of six was reached. A constant log dose increment or decrement of 0.1, corresponding to a dilution factor of 1.26 was used. The ED₅₀ was calculated using the equation:
Log ED₅₀=x_{f}+kd
Where x_{f} is the logarithm of final test dose, d is the log dose increment or decrement, and k is obtained from a table based on maximum likelihood estimates (Dixon, W. J., 1965, J. Amer. Stat. Assoc. 60:967-978). The standard error of log ED₅₀ (s.e.) was estimated using the equation:
s.e.=σ√a
where a equals 0.31 for a nominal sample size of six (Dixon, W. J., 1991 Neuroscience & Biobehavioral Reviews 15:47-50) and a is the population standard deviation, which was assumed to be 0.12 (Bruce, R. D., 1985, Fundamentals & Applied Toxicology 5:151-157).

FVIII coagulation assays and ELISAs—FVIII activity was measured by one-stage clotting assay (Bowie and Owen, 1984, in Disorders of Hemostasis, O. D. Ratnoff and Forbes, C. D., editors, Grune & Stratton, Inc., Orlando, 43-72) as described previously (Doering et al., 2002, J. Biol. Chem. 277:38345-38349) using normal human plasma (FACT) as the standard. FVIII inhibitor titers were measured by a modified Bethesda assay in which fVIII constructs (HBD, R484A/R489A, R484A/R489A/P492A, HP9 or PBD) were added to hemophilia A plasma to a final concentration of 0.8-1.2 units per ml incubated with varying concentrations of inhibitor for 2 hours at 37° C. One Bethesda unit (BU) is defined as the amount of inhibitory activity that produces 50% inhibition of fVIII activity in the one-stage clotting assay. The 50% inhibition point was identified by interpolation using only data points falling within a range of 40-60% inhibition. An average of at least three data points in this range were used for each determination. Because of the smaller volumes required, fVIII activity was measured in the pharmacokinetic study by a chromogenic assay (Coamatic, Chromogenix/Diapharma Group, West Chester, Ohio) according to instructions supplied by the manufacturer.

Antibodies to HBD, R484A/R489A, R484A/R489A/P492A, MBD or HM1 were measured by sandwich ELISA by immobilizing the respective fVIII antigens and using ESH4 and biotinylated-ESH8 as capture and detection antibodies, respectively, as described previously (Lubin et al., 1994, J. Biol. Chem. 269:8639-8641). Absorbance values obtained from seven dilution of test plasma were plotted versus the logarithm of the plasma dilution and the resulting sigmoidal curves were fit to modified version of the 4-parameter logistic fit equation by nonlinear regression using the Levenberg-Marquardt algorithm. The ELISA titer was defined empirically as the dilution of plasma that returns an absorbance value of 0.3 derived from the fitted curves.

Immunodominance of human R484-I508 epitope in hemophilia A mice—To reduce the immunogenecity of human fVIII, two HBD fVIII mutants were developed that contain alanine substitutions of antigenic amino acids in the A2 domain. These mutants, designated R484A/R489A and R484A/R489A/P492A, were compared (FIG. 1) to “wild-type” HBD fVIII in hemophilia A mice using an intravenous immunization protocol described above. Two approaches were used to determine whether hemophilia A mice recognize the R484-I508 A2 sequence that is immunodominant in humans. First, plasmas from the HBD treatment group were tested for reactivity against a hybrid human/porcine fVIII molecule, designated HP9, which is human except for insertion of porcine sequence within the 484-508 segment (FIG. 1). If immune plasmas contain antibodies that recognize the R484-I508 epitope, they should react less well with the HP9 molecule than HBD because of the incomplete cross-reactivity of fVIII inhibitors with human and porcine fVIII. The average Bethesda titers in the HBD treatment group against HBD and B domainless porcine fVIII were 680 and 31 Bethesda units, respectively, corresponding to a cross-reactivity of 4%, demonstrating that porcine fVIII is poorly cross-reactive in this model. FIG. 2A shows paired data comparing the inhibition of HBD and HP9 by individual mouse plasmas in the Bethesda assay. Bethesda titers of most of the pairs were decreased using HP9 as the target antigen compared to HBD. The difference between HP9 and HBD groups was statistically significant (p<0.0001, paired t test). FIG. 2A also shows that all of the plasmas recognized HP9, indicating the presence of inhibitory epitopes directed toward human outside of the R484-I508 A2 epitope.

The immunodominance of the human R484-I508 segment in hemophilia A mice also was tested by a converse experiment in which the same plasma were tested by ELISA for reactivity against a hybrid human/murine fVIII molecule, designated HM1, which is murine except for human R484-I508 segment (FIG. 1). The average ELISA titer against B domainless murine fVIII, MBD, was 19% of that against HBD, demonstrating that plasmas cross-react poorly with murine fVIII, and thus that human/murine hybrid fVIII molecules can be used to study differential antigenicity. The HM1 hybrid would be expected to be more antigenic compared to MBD because of the presence of the antigenic human R484-I508 segment. FIG. 2B shows that ELISA titers of most of the plasmas were increased using HM1 as the target anitgen compared to MBD. The difference between the HM1 and MBD groups was statistically significant (p<0.02, paires t test).

Additionally, in the plasmas from mice immunized with HBD, there was a significant correlation between the reduction in antigenicity of HP9 compared to HBD and the increase in antigenicity of HM1 compared to MBD (FIG. 3). Thus, the degree to which individual mice developed an immune response to the R484-I508 epitope could be detected similarly in both assay systems. This indicates that the reduced antigenicity of HP9 and the increased antigenicity are not artifactual, for example, due to differences in antigen binding to microtiter plates.

Comparative immunogenicity of HBD, R484A/R489A/P492A, R484A/R489A—The hemophilia A mice in the HBD, R484A/R489A, and R484A/R489A/P492A treatment groups received six intravenous injections of 10 μg/kg at 14 day intervals and a final injection of 25 μg/kg body as described in example 1. Plasmas obtained two weeks after the last injection were tested for anti-fVIII antibodies by Bethesda assay and by ELISA. FIG. 4 shows that inhibitor titers were lower in the R484A/R489A/P492A group (p=0.01, Mann-Whitney U test). In contrast, inhibitor levels in the R484A/R489A were not significantly different from the HBD group. Plasmas in the three groups also were tested for anti-fVIII antibodies by ELISA. There was no significant difference between the groups.

In vivo clearance of HBD and R484A/R489A/P492A—The immune response following intravenous injection depends on delivery of the immunogen to the spleen. Thus, instead of being intrinsically less immunogenic than HBD, R484A/R489A/P492A can be cleared more rapidly from the circulation. FIG. 5 shows that the pharmacokinetics of R484A/R489A/P492A and HBD are similar in hemophilia A mice.

Comparative efficacy of HBD and R484A/R489A/P492A—The hemostatic efficacy of R484A/R489A/P492A was measured in hemophilia A mice using a tail vein transection model (Parker and Lollar, (2003) Thromb. Haemost. 89:480-485). The estimated dose of R484A/R489A/P492A that produces 50% survival (ED₅₀) was measured using the up-and-down method (Dixon, W. J., (1965) J. Amer. Stat. Assoc. 60:967-978; Dixon and Massey, (1969) “Sensitivity Experiments,” in Introduction to Statistical Analysis, McGraw Hill, New York, 377-394). The results are shown in Table 2. The method yielded an ED₅₀ of 47.5 units/kg (95% confidence interval, 34.9-64.6 units/kg) for R484A/R489A/P492A. For HBD, and ED₅₀ of 57.7 units/kg (95% confidence interval, 42.4-78.5 units/kg) has been published (Parker and Lollar, (2003) Thromb. Haemost. 89:480-485). The published data for HBD and R484A/R489A/P492A data in the current study were collected contemporaneously on matched littermates. The comparison indicates that the hemostatic efficacy of HBD and R484A/R489A/P492A in this model are indistinguishable.

TABLE 2
Hemostatic efficacy of R484A/R489A/P492A in hemophilia A mice

| Dose | Day | Day | | | | | |
|---|---|---|---|---|---|---|---|
| (units/kg) | 0 | 1 | Day 2 | Day 3 | Day 4 | Day 5 | Day 6 |
| 39.8 | | | X | | | | X |
| 50.1 | | ◯ | | X | | ◯ | |
| 63.1 | ◯ | | | | ◯ | | |

ED₅₀ = 47.5 units/kg*
95% cofidence interval: 34.9-64.6 units/kg*
◯ - Alive
X - Dead

The E16 knockout hemophilia A mice used in this study, along with the genetically similar, phenotypically indistinguishable E17 knockout mice, contained targeted disruptions in the region of the fVIII gene that encodes the A3 domain (Bi et al., (1995) Nat. Genet 10:119-121; Bi et al., (1996) Blood 88:3446-3450). Both strains secrete a non-functional polypeptide containing sequence NH2-terminal to A3 domain, including the A2 domain (Sarkar et al., (2000) Hum. Gene Ther. 11:881-894). In contrast to the murine hemophilia A mice that are available, human hemophilia A is extremely heterogeneous (Tuddenham et al., (1994) Nucleic. Acids. Res. 22:4851-4868). Mutations produce phenotypes that range from no detectable synthesis of fVIII to secretion of partly to completely nonfunctional fVIII. E16 and E17 mice are analogous to hemophilia A patients who secrete nonfunctional fVIII. The E16 mice are not tolerant to the A2 domain despite the presence of truncated circulating fVIII that contains the A2 domain (FIGS. 2 and 3). This may be due to non-native folding of the truncated fVIII polypeptide chain, which prevents induction of tolerance.

An immunogenicity model was used in which fVIII is given intravenously to hemophilia A mice using a dosage schedule that mimics the use of fVIII in human hemophilia A (Qian et al., (1999) Thromb. Haemost. 81:240-244). In this model, hemophilia A mice develop a T cell-dependent antibody response (Qian et al., (1999) Thromb. Haemost. 81:240-244; Wu et al., (2001) Thromb. Haemost. 85:125-133; Reipert et al., (2000) Thromb. Haemost. 84:826-832) that can be inhibited by blockade of the CD28-B7 (Qian et al., (2000) Blood 95:1324-1329) or CD40-CD154 (Qian et al., Eur. J. Immunol. 30:2548-2554; Rossi et al., (2001) Blood 97:2750-2757; Reipert et al., (2002) Thromb. Haemost. 86:1345-1352) co-stimulation pathways. The anti-fVIII antibody response is not isotypically restricted and its distribution is not different from normal mice. (Wu et al., (2001) Thromb. Haemost. 85:125-133; Reipert et al., (2000) Thromb. Haemost. 84:826-832). The effects on immunogenicity observed with the recombinant or recombinant modified fVIII molecules described herein are therefore likely to be comparative to those observed in humans.

### Example 2

### Construction and Evaluation of the A2C2epi1, A2C2epi2 and A2C2epi3 Mutants

Construction of recombinant fVIII mutantcDNAs—The cDNA encoding a human B-domain deleted (HBD) form of fVIII was prepared as described in Doering et al., 2002, J. Biol. Chem. 277:38345-38349. It contains a S F S Q N P P V L K R H Q R linker sequence between the A2 and ap-A3 domains. The A2epi7 cDNA was prepared as described in Example 1. The A2C2epi1, A2C2epi2 and A2C2epi3 cDNAs were prepared by splicing-by-overlap extension mutagenesis using A2epi7 as a template.

Expression and purification of recombinant fVIII molecules—Recombinant fVIII molecules were expressed in baby hamster kidney derived-cells in serum-free medium using the ReNeo expression vector as described previously in Healey et al., 1998, Blood 92:3701-3709. HBD and A2epi7 were isolated using SP-Sepharose Fast Flow and Source Q ion-exchange chromatography essentially as describe previously for HBD in Doering et al., 2002, J. Biol. Chem. 277:38345-38349; and Doering et al., 2002, Thromb. Haemost. 88:450-458. Because of the relatively low yields of A2C2epi1, A2C2epi2 and A2C2epi3 in cell culture, an additional monoS chromatography step was used.

Immunization of hemophilia A mice—FVIII preparations were diluted to 0.17 mg/ml in 0.4 M NaCl, 20 mM HEPES, 5 mM CaCl₂, 0.01% Tween-80 pH 7.4 and stored in small aliquots at −80° C. Samples were diluted were diluted to 4 μg/ml in sterile normal saline immediately prior to injection. Nine- to twelve-week old E16 male or female hemophilia A mice were randomized to five groups of 125. Approximately equal numbers of males and females were in each group. Mice received six injections of 10 μg/kg body weight (˜80 U/kg) at 7 day intervals, followed by a final injection of 25 μg/kg body weight (˜200 U/kg) one week after the sixth dose. Blood was collected into 1/10 volume 3.8% trisodium citrate by terminal cardiac puncture 4 days after the final injection. Samples were placed on ice before centrifugation at 3000×g for 15 minutes at 4° C. to collect plasma.

FVIII coagulation assays and ELISAs—FVIII activity was measured by one-stage clotting assay (Bowie and Owen, 1984, in Disorders of Hemostasis, O. D. Ratnoff and Forbes, C. D., editors, Grune & Stratton, Inc., Orlando, 43-72) as described previously (Doering et al., 2002, J. Biol. Chem. 277:38345-38349) using normal human plasma (FACT) as the standard. In some assays the activated partial thromboplastin time reagent was diluted from the standard concentration. FVIII inhibitor titers were measured by a modified Bethesda assay in which fVIII constructs were added to hemophilia A plasma to a final concentration of 0.8-1.2 units per ml incubated with varying concentrations of inhibitor plasma for 2 hours at 37° C. One Bethesda unit (BU) is defined as the amount of inhibitory activity that produces 50% inhibition of fVIII activity in the one-stage clotting assay. The 50% inhibition point was identified by interpolation using only data points falling within a range of 40-60% inhibition. An average of at least three data points in this range were used for each determination.

Antibodies to HBD, A2epi7, A2C2epi1, A2C2epi2 and A2C2epi3 were measured by sandwich ELISA using immobilized isologous fVIII antigens. Murine monoclonal antibodies ESH4 and biotinylated-ESH8 were used as the capture and detection antibodies, respectively, as described previously (Lubin et al., 1994, J. Biol. Chem. 269:8639-8641). Absorbance values obtained from seven dilution of test plasma were plotted versus the logarithm of the plasma dilution and the resulting sigmoidal curves were fit to modified version of the 4-parameter logistic fit equation by nonlinear regression using the Levenberg-Marquardt algorithm. The ELISA titer was defined empirically as the dilution of plasma that returns an absorbance value of 0.3 derived from the fitted curves.

Construction, expression and purification of recombinant proteins—The C2 domain of fVIII contains three hydrophobic loops that comprise the binding site for phospholipid membranes (Pratt et al., (1999) Nature 402:439-442). Loops 1 and 2, corresponding to amino acids M2199/F2200 and L2251/L2252 in human fVIII are antigenic (Barow et al., (2001) Blood 97:169-174). Amino acids were selected for replacement of human residues at loops 1 and 2 based on the sequences of murine, canine, porcine, chicken and pufferfish (Fugu rubripes) fVIII (Table 3). The rationale for making substitutions with animal amino acids was that phospholipid membrane binding may have been preserved during evolutionary drift, thereby avoiding loss of function due to the mutations. The amino acid replacements for the A2C2epi1, A2C2epi2 and A2C2epi3 constructs is shown in FIG. 6.

TABLE 3
Amino acids at phopholipid binding loops 1 and 2 in fVIII

| Amino Acid Residue | |
|---|---|
| Loop 1 | Loop 2 |

| Species | 2199 | 2200 | 2251 | 2252 |
|---|---|---|---|---|
| Human | M | F | L | L |
| Mouse | M | F | L | F |
| Dog | M | L | L | L |
| Pig | I | F | L | L |
| Chicken | I | F | V | F |
| Pufferfish | L | L | L | L |

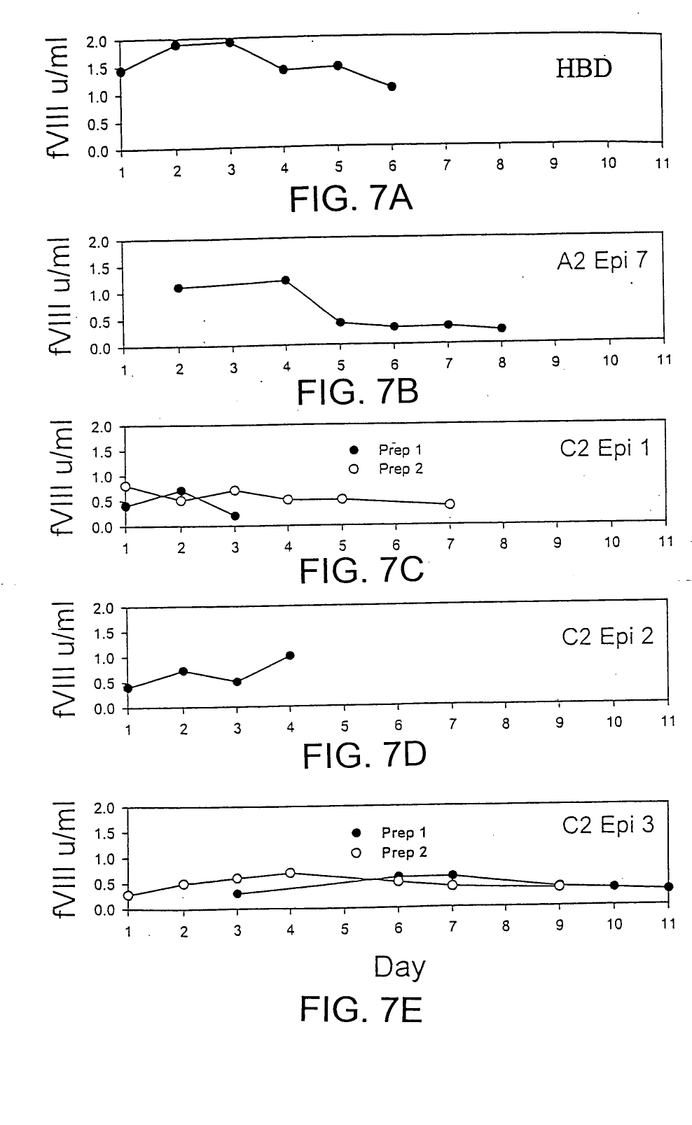

FVIII cDNAs were transfected into BHK-derived cells and selected for neomycin resistance. The highest expressing clones for HBD, A2epi7, A2C2epi1, A2C2epi2 and A2C2epi3 were expanded into triple flasks for production of fVIII. Cell culture supernatants from each construct were collected every 24 or 48 h for three to eleven days and pooled. The relative expression of A2epi7, A2C2epi1, A2C2epi2 and A2C2epi3 was two- to four-fold lower than HBD (FIG. 7). Two production runs of A2C2epi1 and A2C2epi3 were done to obtain sufficient material for the study.

The fVIII constructs were isolated by ion-exchange chromatography. Because the lower yields of A2C2epi1, A2C2epi2 and A2C2epi3 equate to a larger amount of impurities in the starting material, an additional mono S ion-exchange chromatography step was added. Purification tables for the constructs are shown in Table 4. The yields of the different constructs were similar through the Source Q chromatography step. The overall yields of A2C2epi1 and A2C2epi3 were lower because of the additional mono S chromatography step. The activation quotients (AQs) for the preparations ranged from 11 to 23. The AQ of A2C2epi1 and A2C2epi3 were somewhat lower than HBD, the significance of which is not known. The specific coagulant activity (U/A₂₈₀) of A2C2epi1 was lower than HBD. The specific activities of the other fVIII constructs were similar to HBD with the exception of A2C2epi2, which was approximately 25% higher.

SDS-page analysis of the preparation is shown in FIG. 8. The preparations were highly purified and appeared to be similar in composition. All contained a slight amount of a low molecular weight contaminant (marked by “*”) and a small amount of single chain fVIII. All of the preparations were cleaved by thrombin (factor IIa) to produce characteristic A1, A2 and thrombin-cleaved light chain subunits.

TABLE 4
Purification tables for HBD, A2epi7, A2C2epi1, A2C2epi2 and A2C2epi3

| | | | | | | | | | |
|---|---|---|---|---|---|---|---|---|---|
| | | | Total | | | Units/ | | % | Fold |
| Sample | Volume† | A₂₈₀ | A₂₈₀ | Activity‡ | Units | A₂₈₀ | AQ | Yield | Pur. |

HBD

| | | | | | | | | | |
|---|---|---|---|---|---|---|---|---|---|
| Media | 12000 | 2.911 | 34927 | 1.50 | 18000 | 0.5 | 29 | 100% | 1 |
| SP- | 285 | 0.030 | 8.408 | 43.4 | 12369 | 1471 | 16 | 69% | 2855 |
| Sepharose | | | | | | | | | |
| pool | | | | | | | | | |
| Source Q | 5.6 | 0.398 | 2.229 | 1287.0 | 7207 | 3234 | 21 | 40% | 6275 |
| Pool | | | | | | | | | |

A2epi7

| | | | | | | | | | |
|---|---|---|---|---|---|---|---|---|---|
| Media | 12,600 | 2.802 | 35305 | 0.66 | 8316 | 0.2 | 9.4 | 100% | 1 |
| SP- | 114 | 0.060 | 6.840 | 43.0 | 4902 | 717 | 25 | 59% | 3043 |
| Sepharose | | | | | | | | | |
| pool | | | | | | | | | |
| Source Q | 3.8 | 0.162 | 0.616 | 590.0 | 2242 | 3642 | 20 | 27% | 15462 |
| Pool | | | | | | | | | |

A2C2epi1 #1

| | | | | | | | | | |
|---|---|---|---|---|---|---|---|---|---|
| Media | 8,400 | 2.802 | 23537 | 0.33 | 2772 | 0.1 | 7 | 100% | 1 |
| SP- | 148 | 0.028 | 4.144 | 4.0 | 592 | 143 | 16 | 21% | 1213 |
| Sepharose | | | | | | | | | |
| pool | | | | | | | | | |
| Source Q | 10 | 0.081 | 0.810 | 65.00 | 650 | 802 | 17 | 23% | 8025 |
| Pool | | | | | | | | | |
| Mono S Pool | 3.2 | 0.109 | 0.349 | 211.0 | 675 | 1936 | 10 | 24% | 19358 |

A2C2epi1 #2

| | | | | | | | | | |
|---|---|---|---|---|---|---|---|---|---|
| Media | 10,000 | 2.800 | 28,000 | 0.15 | 1450 | 0.1 | 6 | 100% | 1 |
| SP- | 202 | 0.033 | 6.666 | 5.1 | 1030 | 155 | 11 | 71% | 2984 |
| Sepharose | | | | | | | | | |
| pool | | | | | | | | | |
| Source Q | 4.8 | 0.139 | 0.667 | 105.0 | 504 | 755 | 9 | 35% | 14587 |
| Pool | | | | | | | | | |
| Mono S Pool | 3.2 | 0.023 | 0.074 | 72.4 | 232 | 3148 | 23 | 16% | 60535 |

A2C2epi2

| | | | | | | | | | |
|---|---|---|---|---|---|---|---|---|---|
| Media | 8,000 | 2.802 | 22,416 | 0.73 | 5840 | 0.3 | 12 | 100% | 1 |
| SP- | 162 | 0.019 | 3.013 | 25.2 | 4082 | 1355 | 17 | 70% | 5200 |
| Sepharose | | | | | | | | | |
| pool | | | | | | | | | |
| Source Q | 10 | 0.102 | 1.020 | 240.00 | 2400 | 2353 | 24 | 41% | 9031 |
| Pool | | | | | | | | | |
| Mono S Pool | 3.1 | 0.180 | 0.558 | 790.0 | 2449 | 4389 | 23 | 42% | 14630 |

A2C2epi3 #1

| | | | | | | | | | |
|---|---|---|---|---|---|---|---|---|---|
| Media | 8,400 | 2.802 | 23537 | 0.27 | 2285 | 0.1 | 10 | 100% | 1 |
| SP- | 175 | 0.018 | 3.150 | 4.4 | 770 | 244 | 13 | 34% | 2518 |
| Sepharose | | | | | | | | | |
| pool | | | | | | | | | |
| Source Q | 9 | 0.070 | 0.630 | 70.6 | 635 | 1009 | 10 | 28% | 10390 |
| Pool | | | | | | | | | |
| Mono S Pool | 3.2 | 0.053 | 0.170 | 152.0 | 486 | 2868 | 15 | 21% | 28679 |

A2C2epi3 #2

| | | | | | | | | | |
|---|---|---|---|---|---|---|---|---|---|
| Media | 12,500 | 2.802 | 35025 | 0.22 | 2800 | 0.1 | 8 | 100% | 1 |
| SP- | 148 | 0.041 | 6.068 | 11.0 | 1628 | 268 | 9 | 58% | 3356 |
| Sepharose | | | | | | | | | |
| pool | | | | | | | | | |
| Source Q | 8.8 | 0.198 | 1.742 | 158.0 | 1390 | 798 | 12 | 50% | 9982 |
| Pool | | | | | | | | | |
| Mono S Pool | 3.2 | 0.111 | 0.355 | 283.0 | 906 | 2550 | 11 | 16% | 31869 |

†milliliters
‡units/milliliter

Comparative immunogenecity A2epi7, A2C2epi1, A2C2epi2 and A2C2epi3 in hemophilia A mice—The immunogenicity of HBD, A2epi7, A2C2epi1, A2C2epi2 and A2C2epi3 were compared in a randomized trial in hemophilia A mice. Antibodies to the immunogens were measured by Bethesda assay (FIG. 9) and by anti-fVIII ELISA (FIG. 10). The isologous form of fVIII was used as the antigen. The Bethesda titers of the A2epi7, A2C2epi1 and A2C2epi3 were significantly less than the HBD group (p<0.0001, p=0.004, and p<0.0001, respectively, Mann-Whitney U test). In contrast, the immunogenicity of the A2C2epi2 group was not significantly lower than the HBD group. Additionally, the Bethesda titer of the A2C2epi3 group was significantly lower than the A2epi7 group (p=0.009). The average Bethesda titer of the A2C2epi3 group was only 6.8, compared to average titers of 290, 62, 110 and 270 for the HBD, A2epi7, A2C2epi1 and A2C2epi2 groups, respectively.

By ELISA, the immunogenicity rank order also was HBD>A2epi7>A2C2epi3. The results of all pair wise comparisons within this rank order were statistically significant (p<0.01). However, in contrast to the Bethesda assay, the titer of A2C2epi1 in the ELISA assay was not significantly less than HBD. The immunogenicity of A2C2epi3 was profoundly depressed in the ELSA with only 3 of 25 mice testing positive.

Antigenicity of HBD in A2C2epi3-immunized mice—The Bethesda and ELISAs were performed using the isologous form of fVIII as the antigen. For example, in A2C2epi3-immunized mice, purified A2C2epi3 was used to reconstitute hemophilia A plasma in the Bethesda assay and was used to coat ELISA plates. To guard against the possibility that A2C2epi3 immunized mice might have developed a strong cross-reactive response to human fVIII, ten plasma samples were randomly selected from the A2C2epi3 group and tested by Bethesda assay using hemophilia A plasma reconstituted with A2C2epi3 or HBD. Table 5 shows that the immune response to HBD was less than or equal to A2C2epi3 in all of the mice. This confirms that the plasmas in the A2C2epi3 group are poorly reactive with human fVIII inhibitory epitopes.

TABLE 5
Bethesda titers of HBD in A2C2epi3 immunized mice

ANTIGEN

| Mouse ID | A2C2epi3 | HBD |
|---|---|---|
| 2139-3 | 13 | <1.5 |
| 2134-1 | 13 | 13 |
| 2169-1 | 38 | 19 |
| 2123-2 | <1.5 | <1.5 |
| 2125-4 | <1.5 | <1.5 |
| 2122-4 | <1.5 | <1.5 |
| 2121-3 | <1.5 | <1.5 |
| 2124-2 | <1.5 | <1.5 |
| 2129-3 | <1.5 | <1.5 |
| 2141-1 | <1.5 | <1.5 |

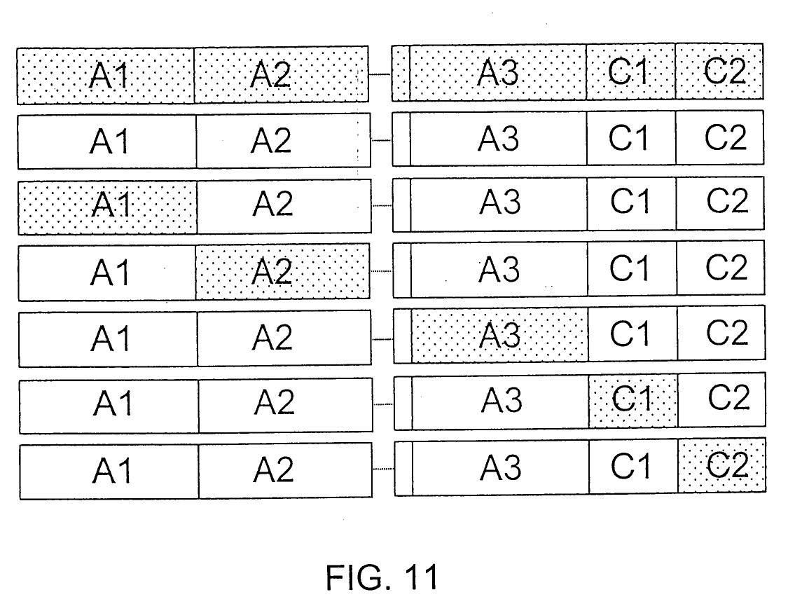

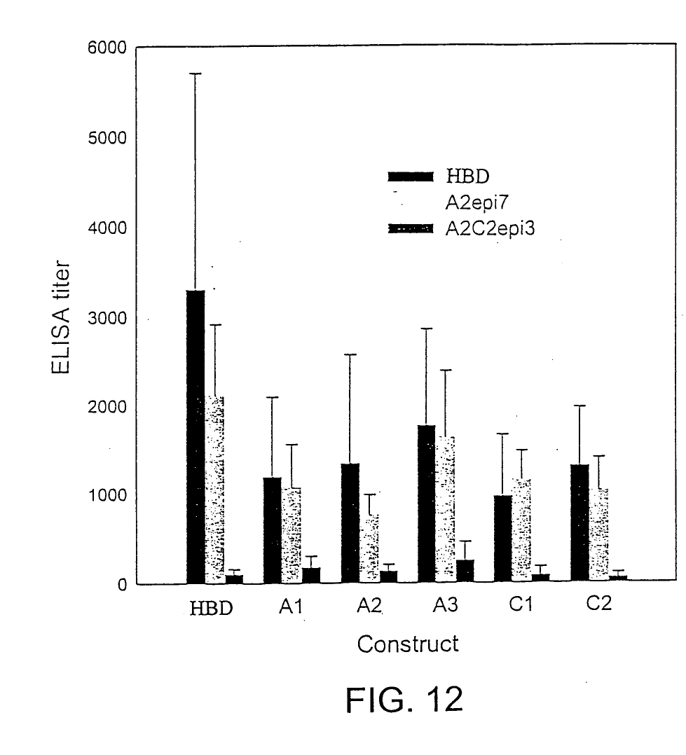

Domain specificity of anti-fVIII antibodies in hemophilia A mice immunized with HBD, A2epi7 and A2C2epi3—Single human domain hybrid human/porcine fVIII constructs (FIG. 11) were expressed in baby hamster kidney-derived cells, purified and immobilized on microtiter plates. ELISA titers from ten plasmas from each of the HBD and A2epi7 groups were measured. Only five plasmas in the A2C2epi3 group had significantly positive ELISA titers and were selected for study. FIG. 12 shows antibodies from plasmas from the HBD and A2epi7 groups recognize all of the human domains that were studied (A1, A2, A3, C1 and C2).

There was a marked reduction in total anti-A2C2epi3 antibodies by ELISA (FIG. 10). Consistent with this, there was a reduction in antibodies to the A1, A2, A3, C1 and C2 domains by domain-specific ELISA (FIG. 12). If the immune system indeed is less able to respond to A2C2epi3, this indicates that anti-fVIII antibodies may develop by epitope spreading, which has been described for other immunogens (McKluskey et al., (1998) Immunol. Rev. 164:209-229). Thus, in the absence of initial strong recognition of the C2 domain the overall immune response may be blunted. There was also a trend toward a lower anti-A2 titer in the A2epi7 group (p=0.11, Mann-Whitney U test). A hybrid human/porcine fVIII molecule that contains the A2epi7 triple mutation can be constructed in order to determine whether the sequence bounded by residues 484-508 is recognized less strongly in A2epi7-immunized mice.

In vitro recovery of fVIII activity—The low immunogenicity of A2C2epi3 and A2epi7 could be due to denaturation of the proteins in murine hemophilia A plasma, resulting in poor delivery of the immunogen to the immune system. As a first step to test this possibility, the in vitro recovery of fVIII activity of the test constructs was measured. Data are available from the Bethesda assays, which were done by reconstruction of human hemophilia A plasma with the isologous constructs. After reconstitution, plasmas were assayed to determine the recovery of fVIII based in the activity of the purified material. This was done on at least eight occasions for each construct. FIG. 11 shows the recovery of activity was within the expected range for all of the constructs.

Phospholipid-dependent activated partial thromboplastin times of A2C2epi3 and HBD—Because A2C2epi3 contains mutations at sites known to be important for the binding of fVIII to phospholipid, it is possible that it is defective with respect to this function. The specific activities of two independent preparations of A2C2epi3 were 2870 and 2550 U/A₂₈₀, compared to 3230 U/A₂₈₀ for HBD. These values are considered within experimental error, indicating that A2C2epi3 is fully functional. Activated partial thromboplastin (aPTT)-based fVIII assays are performed at high concentrations of phospholipid. This may drive the equilibrium of forms of activated fVIII that have lower affinities for phospholipid to the bound site. To test this possibility, the aPTTs were performed on human hemophilia A plasmas reconstituted with HBD or A2C2epi3 at various dilutions of aPTT reagent, which contains phospholipid and a contact activation surface (FIG. 12). Dilutions of HBD and A2C2epi3 were made that result in equal clotting times using undiluted aPTT reagent. As the concentrations of aPTT reagent decreased below 0.5 of normal, there was an increase in clotting time for both HBD and A2C2epi3, indicating that phospholipid was a limiting component in the assay. There was a slight increase in the clotting time of A2C2epi3 compared to HBD at dilutions of aPTT reagent up to four-fold and then a more pronounced increase at higher dilutions. This result indicates that A2C2epi3 may have a lower affinity for phospholipid membranes than HBD.

While the invention has been described with certain preferred embodiments, it is understood that the preceding description is not intended to limit the scope of the invention. It will be appreciated by one skilled in the art that various equivalents and modifications can be made to the invention shown in the specific embodiments without departing from the spirit and scope of the invention. All references are incorporated herein to the extent not inconsistent.

1. A modified human fVIII comprising an immunoreactivity reducing amino acid substitution at each of positions number 484, 489 and 492 as set forth in SEQ ID NO: 2.

2. The modified human fVIII of claim 1 wherein each immunoreactivity reducing amino acid substitution is selected independently from the group consisting of alanine, methionine, leucine, serine, and glycine.

3. The modified human factor VIII of claim 2 wherein each immunoreactivity reducing amino acid substitution is alanine.

4. The modified human factor VIII of claim 2 wherein each immunoreactivity reducing amino acid substitution is the corresponding amino acid from porcine fVIII as set forth in SEQ ID NO: 3.

5. The modified human fVIII of claim 1 comprising a partial deletion of the B domain.

6. The modified human fVIII of claim 1 wherein the partial B-domain deletion consists of deletion of amino acids 746-1639 as set forth on SEQ ID NO: 2.

7. The modified human fVIII of claim 1 wherein said modified fVIII is a continuous single polypeptide.

8. The modified human fVIII of claim 1 wherein said modified fVIII is a A1/A2/A3-C1-C2 heterotrimer.

9. The modified human fVIII of claim 1 wherein said modified fVIII is a A1-A2/A3-C1-C2 heterodimer.

10. The modified human factor VIII of claim 1 wherein leucine is substituted for methionine 2199, leucine is substituted for phenylalanine 2200, valine is substituted for leucine 2251, and phenylalanine is substituted for leucine 2252 as set forth on SEQ ID NO: 2.

11. A pharmacological composition comprising the modified human fVIII of claim 1.

12. A method of managing a hemophilic patient comprising administering to said patient the pharmacological composition of claim 11.

13. DNA encoding a modified human fVIII comprising an immunoreactivity reducing amino acid substitution at each of positions number 484, 489 and 492 as set forth in SEQ ID NO: 2.

14. The DNA of claim 13 wherein each immunoreactivity reducing amino acid substitution is selected independently from the group consisting of alanine, methionine, leucine, serine, and glycine.

15. The DNA of claim 14 wherein each immunoreactivity reducing amino acid substitution is alanine.

16. The DNA of claim 14 wherein each immunoreactivity reducing amino acid substitution is the corresponding amino acid from porcine fVIII as set forth in SEQ ID NO: 3.

17. The DNA of claim 13 wherein leucine is substituted for methionine 2199, leucine is substituted for phenylalanine 2200, valine is substituted for leucine 2251, and phenylalanine is substituted for leucine 2252 as set forth in SEQ ID NO: 2.

18. A method of producing a modified fVIII by expressing the DNA of claim 17 in a host cell.

19. The method of claim 18 wherein said DNA is a continuous DNA sequence.

20. A modified human fVIII comprising an amino acid substitution each of positions number 2199, 2200, 2251 and 2252 as set forth in SEQ ID NO: 2, wherein leucine is substituted for methionine 2199, leucine is substituted for phenylalanine 2200, valine is substituted for leucine 2251, and phenylalanine is substituted for leucine 2252.

21. The modified human fVIII of claim 20 comprising a partial deletion of the B domain.

22. The modified human fVIII of claim 21 wherein the partial B-domain deletion consists of deletion of amino acids 746-1639 as set forth on SEQ ID NO: 2.

23. The modified human fVIII of claim 20 wherein said modified fVIII has reduced immunogenicity as compared to unmodified fVIII.

24. A pharmacological composition comprising the modified human fVIII of claim 20.

25. A method of managing a hemophilic patient comprising administering to said patient the pharmacological composition of claim 24.

26. DNA encoding a modified human fVIII comprising modified human fVIII wherein leucine is substituted for methionine 2199, leucine is substituted for phenylalanine 2200, valine is substituted for leucine 2251, and phenylalanine is substituted for leucine 2252 as set forth in SEQ ID NO: 2.

27. A method of producing a modified fVIII by expressing the DNA of claim 26 in a host cell.

28. The method of claim 27 wherein said DNA is a continuous DNA sequence.

29. A hybrid fVIII comprising: the A1, A2, A3 and C1 domains of human fVIII, having an immunoreactivity reducing amino acid substitution at each of positions number 484, 489 and 492 as set forth in SEQ ID NO: 2; and the C2 domain of a non-human fVIII.

30. The hybrid fVIII of claim 29 comprising the C2 domain of porcine fVIII as set forth in SEQ ID NO: 3.

31. The hybrid fVIII of claim 29 wherein each immunoreactivity reducing amino acid substitution is alanine.

32. DNA encoding a hybrid fVIII comprising: the A1, A2, A3 and C1 domains of human fVIII, having an immunoreactivity reducing amino acid substitution at each of positions number 484, 489 and 492 as set forth in SEQ ID NO: 2; and the C2 domain of a non-human fVIII.

33. The DNA of claim 32 wherein said hybrid fVIII comprises the C2 domain of porcine fVIII as set forth in SEQ ID NO: 3.

34. A hybrid fVIII comprising:
(a) the A1, A3, C1 and C2 domains of human fVIII, having an amino acid substitution each of positions number 2199, 2200, 2251 and 2252 as set forth in SEQ ID NO: 2, wherein leucine is substituted for methionine 2199, leucine is substituted for phenylalanine 2200, valine is substituted for leucine 2251, and phenylalanine is substituted for leucine 2252; and
(b) the A2 domain of a non-human fVIII.

35. The hybrid fVIII of claim 34 comprising the A2 domain of porcine fVIII as set forth in SEQ ID NO: 3.

36. DNA encoding a hybrid fVIII comprising:
(a) the A1, A3, C1 and C2 domains of human fVIII, having an amino acid substitution each of positions number 2199, 2200, 2251 and 2252 as set forth in SEQ ID NO: 2, wherein leucine is substituted for methionine 2199, leucine is substituted for phenylalanine 2200, valine is substituted for leucine 2251, and phenylalanine is substituted for leucine 2252; and
(b) the A2 domain of a non-human fVIII.

37. The DNA of claim 37 wherein said hybrid fVIII comprises the A2 domain of porcine fVIII as set forth in SEQ ID NO: 3.

38. A method of identifying a modified fVIII having reduced immunogenicity or antigenicity comprising the steps:
(a) injecting at least one dose of said modified fVIII into a first group of animal test subjects;
(b) injecting at least one dose of an unmodified fVIII or a fVIII with known antigenic and/or immunogenic properties into a second group of animal test subjects; and
(c) using a diagnostic assay to compare the inhibitory antibodies produced by said first group of animal test subjects with inhibitory antibodies produced by said second group of animal test subjects.

39. The method of claim 38 wherein said animals are mice.
